(19) United States
(12) Patent Application Publication
(10) Pub. No.: US 2025/0309306 A1
(43) Pub. Date: Oct. 2, 2025

(54) Cationic Viologen Derivatives and Use Thereof in Redox Flow Batteries

(71) Applicant: The Governing Council of the University of Toronto, Toronto (CA)

(72) Inventors: Andrés AGUILAR GRANDA, Toronto (CA); Robert Alexander BATEY, Toronto (CA); Yang CAO, Toronto (CA); Tatsuya Robert Seamus CORLETT, Toronto (CA); Liam FORTIER, Mississauga (CA); Han HAO, Toronto (CA); Lars Johan Kjell JORNER, Zurich (CH); Cher Tian SER, Toronto (CA); Marta SKRETA, Toronto (CA); Alan ASPURU-GUZIK, Toronto (CA)

(73) Assignee: The Governing Council of the University of Toronto, Toronto (CA)

(21) Appl. No.: 19/094,206

(22) Filed: Mar. 28, 2025

Related U.S. Application Data
(60) Provisional application No. 63/571,244, filed on Mar. 28, 2024.

(51) Int. Cl. H01M 8/18; C07D 223/00; C09K 9/02; H01M 8/08

(52) U.S. Cl. CPC H01M 8/18; C07D 223/00; C09K 9/02; H01M 8/08; C09K 2211/1044

(57) ABSTRACT

The present application provides cationic viologen derivatives, referred to as transquats, and compositions and uses thereof. The transquats undergo cycles of reduction and oxidation processes in water and are useful in various applications, for example, as electrolytes in Aqueous Redox Flow Batteries (ARFBs). The transquat compounds of the present application have the structure of Formula I

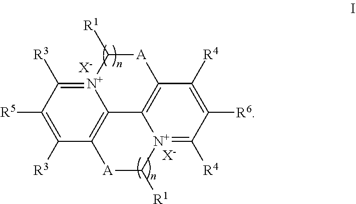

### CROSS-REFERENCE TO RELATED APPLICATION

This application claims priority to U.S. Provisional Application No. 63/571,244, filed Mar. 28, 2024, which is hereby incorporated by reference in its entirety.

### FIELD OF THE INVENTION

The present application pertains to the field of organic functional materials and devices. More particularly, the present application relates to cationic viologen derivatives, methods of preparation thereof and uses and applications thereof, such as in aqueous redox flow battery electrolytes.

### BACKGROUND

Concerns regarding the environmental consequences of using fossil fuels as energy sources have led to the increasing prominence of renewable energy systems (e.g., solar-and wind-based systems). However, the intermittent nature of such renewable energy sources makes it difficult to fully integrate these energy sources into electrical power grids and distribution networks. A solution to this problem may be large-scale electrical energy storage systems, which are also vital for the smart grid and distributed power generation development. Such energy storage systems are also critical to effective electrification of on-ground transportation, as the replacement of traditional combustion engines with hybrid, plug-in hybrid, and pure electric vehicles (EVs), has further increased the need for electricity sources that do not rely on fossil fuels. Challenges to the widespread implementation of electrical energy storage systems include cost, reliability and safety, equitable regulatory environments, and industry acceptance. The development of energy storage technologies capable of resolving these challenges is critical. Redox-flow batteries (RFBs)—first developed by NASA during the energy crisis of the 1970s and recently receiving renewed attention—are among the most promising scalable energy storage technologies.

RFBs are electrochemical systems that can repeatedly store and convert electrical energy to chemical energy and vice versa as needed. Redox reactions are employed to store energy in the form of a chemical potential in liquid electrolyte solutions that flow through a battery comprising electrochemical cells during charge and discharge. The stored electrochemical energy can be converted to electrical energy upon discharge with concomitant reversal of the opposite redox reactions.

RFBs usually include a positive electrode and a negative electrode in cells separated by an ion-exchange membrane, and circulating positive and negative electrolyte flow streams, generally referred to as the “posolyte” and “negolyte”, respectively. Energy conversion between electrical energy and chemical potential occurs instantly at the electrodes, once the electrolyte solutions begin to flow through the cell. During discharge, electrons are released via an oxidation reaction from a high chemical potential state at the anode of the battery and subsequently move through an external circuit. Finally, the electrons are accepted via a reduction reaction at a lower chemical potential state at the cathode of the battery. Redox-flow batteries can be recharged by inversing the flow of the redox fluids and applying current to the electrochemical reactor.

The capacity and energy of redox flow batteries is determined by the total amount of redox-active species for a set system available in the volume of electrolyte solution, whereas their current (power) depends on the number of atoms or molecules of the active chemical species that are reacted within the redox flow battery cell as a function of time. Redox-flow batteries have the advantage that their capacity (energy) and their current (power) can be readily separated, and therefore readily up-scaled. Thus, capacity (energy) can be increased by increasing the number or size of the electrolyte tanks whereas the current (power) is controlled by controlling the number and size of the current collectors. Since energy and power of RFB systems are independent variables, RFBs are inherently suitable for large applications, since they scale-up in a more cost-effective manner than other battery types.

The current state-of-the-art redox flow batteries are vanadium flow batteries and, to a lesser extent, zinc bromide batteries. Both RFBs rely on the chemistries of metals that operate under extreme conditions (i.e., concentrated acid or liquid bromine). Additionally, the chemistries associated with these types of RFBs determine the fundamental properties of these batteries, which are difficult to fine-tune. Research is ongoing to replace vanadium species (and other inorganic electrolytes) with more sustainable alternatives. Furthermore, toxicity and associated health and environmental risks of inorganic redox materials further limit the applicability of inorganic RFBs for energy storage.

In view of the disadvantages of RFBs based on inorganic redox species, aqueous organic RFBs have become the focus of recent research and development. Organic redox active species for large-scale use in redox flow batteries should preferably be inexpensive, have high water solubility and redox potential, and exhibit fast electrode kinetics. To date, quinone, anthraquinone, benzoquinone, alloxazine, phenazine and fluorenone derivatives (among others) have been explored as organic redox-active species for use in RFBs. However, these compounds are costly and typically require elaborate manufacture, which severely limits their broad-range, large-scale employment. Furthermore, most organic molecules suitable for use in RFBs face solubility issues in water. Low water solubility in water leads to a low energy density and less competitive economics.

Viologens are redox-active bipyridinium derivatives that undergo two, stepwise, one-electron reductions. These compounds have excellent electron-accepting capability and have been broadly applied in various fields, such as, but not limited to, supermolecule assembly applications and energy storage. Viologens are also known for their drastic colors in reduced states and, consequently, have been used in electrochromic materials.

Most viologens fall into the paraquat (N,N′-dialkyl-4,4′-bipyridinium) and diquat (N,N′-dialkyl-2,2′-bipyridinium) families, although some variation exists with the two pyridinium rings further separated by a p-conjugated bridge.(1) The most widely produced viologens are paraquat dichloride and diquat dibromide in hydrated form or aqueous solutions. Derivation of these two families to date has mostly been in the form of different counter-ions, and variation in substitution patterns on the aromatic rings.

Currently known viologens are known to decompose under specific conditions. The di-cations are unstable under strongly basic conditions; the reduced species are prone to hydrolysis, or oxidation by redox-active species, in the presence of dioxygen. The 1-e reduced radical cations are also known to dimerize or react with other chemicals in the solution. Moreover, the redox potentials of viologens are strongly limited by the substitutions on the aromatic rings. Although it is known that the torsion of the N—C—C—N backbone can result in more negative redox potentials, which is beneficial for application in flow batteries, it is unclear how to achieve high torsion angles without destabilizing the final structure, and its chemical and electrochemical stabilities.

Viologen derivatives have been utilized in RFBs previously, but with only limited success, largely due to their instability. (2) Unlike neutral structures, such as quinone, fluorenone, and azo-aromatics, viologens are intrinsically ionic and highly soluble in water because of their double cationic charge. However, the electrochemical properties of viologen electrolytes, most of which are paraquats, are not very tunable. For instance, the redox potential of paraquats falls into a narrow band around −0.4 V vs SHE.

Thus, there remains a need for alternative electroactive redox materials and for improved redox flow battery chemistries and systems. Preferably, such electrolytes further provide for a high energy density, a high operating potential, increased cell output voltage and extended lifetime.

The above information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present application is to provide cationic viologen derivatives and compositions and uses thereof, for example, as electrolytes in redox flow batteries. In accordance with one aspect, the present application provides a class of ionic organic molecules, referred to herein as transquats, that undergo cycles of reduction and oxidation (redox) processes in water. These transquat compounds are useful as electrolytes in Aqueous Redox Flow Batteries (ARFBs) because they are relatively low-cost, easy to prepare, redox-reversible, and/or can reach high power and energy densities due to higher cell potential and higher water solubility than other, previously known, organic electrolytes. Alterations in the chemical substituents included in the present transquat compounds can change or attenuate their cell potential and redox reversibility, whereas both the presence of the di-cation and selection of the counterions impact the water solubility of these compounds. Accordingly, the transquat compounds of the present application are tunable for various applications.

In accordance with an aspect of the present application, there is provided having the structure of Formula I

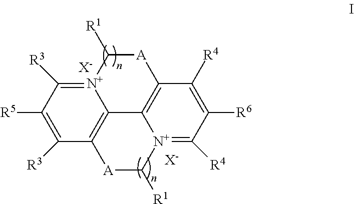

wherein:
   each A is CHR², NR², O, or SOₘ, where m is 0, 1 or 2;
   each R¹ and R² is independently H, optionally substituted C₁-C₆ alkyl, or optionally substituted C₁-C₆ alkoxy, optionally substituted di-alkylamino, optionally substituted di-arylamino, optionally substituted alkyl-arylamino, or a glycol side chain (such as ethylene glycol, propylene glycol, poly(ethylene glycol, or poly(propylene glycol);
   each R³, R⁴, R⁵, and R⁶ is independently H, optionally substituted C₁-C₆ alkyl, or optionally substituted C₁-C₆ alkoxy, optionally substituted di-alkylamino, optionally substituted di-arylamino, optionally substituted alkyl-arylamino, or a glycol side chain (such as ethylene glycol, propylene glycol, poly(ethylene glycol), or poly(propylene glycol);
   each X⁻ is independently a counterion, such as a halide (e.g., Cl⁻ or Br⁻); and
   each n is independently 0, 1 or 2.

In some embodiments, the transquat compound of the present application has the structure of Formula Ia, Ib, Ic, or Id

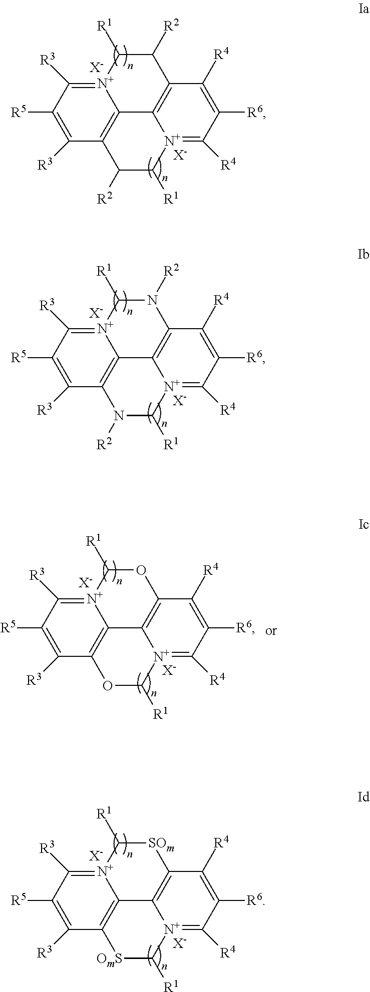

In some embodiments, the transquat compound has the structure of Formula Ia or Ib and each R² is independently H, optionally substituted C₁-C₆ alkyl, or optionally substituted C₁-C₆ alkoxy, or wherein each R² is H.

In some embodiments each R¹ is independently H, optionally substituted C₁-C₆ alkyl, or optionally substituted C₁-C₆ alkoxy, or wherein each R¹ is H.

In some embodiments one or more of each R³ and R⁵ and/or one or more of each R⁴ and R⁶ is an alkyl, such as methyl, ethyl or propyl; and/or one or more of each R³ and R⁵ and/or one or more of each R⁴ and R⁶ is an alkoxy, such as methoxy, ethoxy or propoxy.

In some embodiments, the transquat compound is a salt of one of the following:

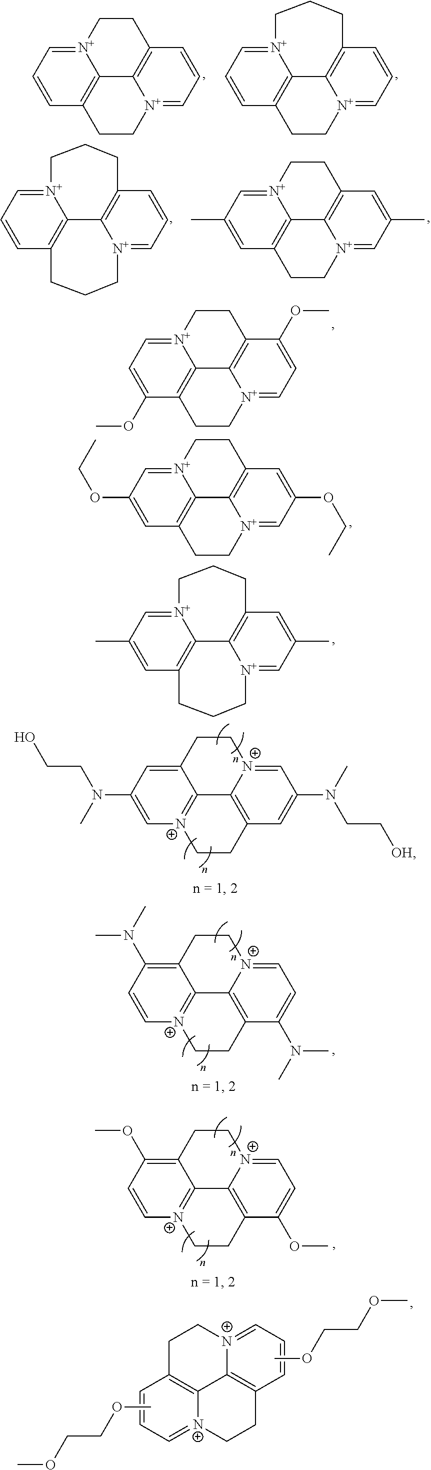

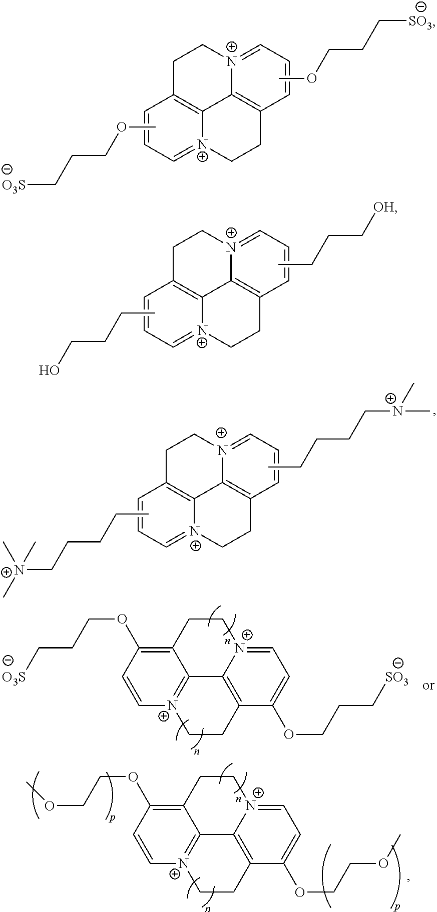

wherein each n is independently 1 or 2, and wherein

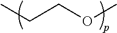

represents an ethyleneoxy moiety when p is 1 or a poly(ethylene glycol) moiety when p is greater than 1.

In accordance with another aspect of the present application, there is provided an electrolyte composition comprising one or more transquat compound as defined herein and water. In accordance with a related aspect of this application, there is provided a method of using the transquat compounds as described herein as a negative electrolyte.

In accordance with another aspect, there is provided a redox flow battery comprising: (a) a negative electrode; (b) a first redox active composition comprising one or more transquat compound as described herein as a negative electrode electrolyte, the negative electrode electrolyte contacting the negative electrode; (c) a positive electrode; (d) a second redox active composition comprising a positive electrode electrolyte contacting the positive electrode; and (d) an ion selective membrane interposed between the negative electrode and the positive electrode.

In accordance with another aspect of the present application, there is provided an electrochromic material comprising one or more transquat compound as described herein, which functions to providing a reversible colour change in all or a portion of the electrochromic material. Optionally, the electrochromic material is a window glass (e.g., a smart window), a device display, a reflective blind, a sensor, a mirror, an eyeglass lens, or the like.

### BRIEF DESCRIPTION OF FIGURES

For a better understanding of the application as described herein, as well as other aspects and further features thereof, reference is made to the following description which is to be used in conjunction with the accompanying drawings, where:

FIG. 1 depicts an overlay of cyclic voltammograms of a transquat according to one embodiment of the present application, a diquat-annulated and a diquat.

FIG. 2 schematically depicts the components of a redox flow battery.

FIG. 3 shows the X-ray crystal structure for the PF₆⁻ salt of 5,6,11,12-tetrahydrodipyrido[3,2,1-de:3′,2′,1′-ij][1,5]naphthyridine-4,10-diium.

FIG. 4 shows the X-ray crystal structure for the OTf⁻ salt of 4,5,6,10,11,12-hexahydro-3a,9a-diazadibenzo[ef,kl]heptalene-3a,9a-diium dichloride.

FIG. 5 (a) is a diagram of a typical three-electrode voltammetry setup; where A=Ammeter, V=Voltmeter, C=Counter electrode, R=Reference electrode, W=Working electrode; and (b) is a photograph of the three-electrode setup employed in the Examples.

FIG. 6 schematically depicts an experimental method used for quantitative water solubility determination.

FIGS. 7 and 8 depict square wave voltammetry, cyclic voltammetry and CV integration for two transquat compounds according to specific embodiments of the present application.

FIGS. 9 and 10 depict square wave voltammetry, cyclic voltammetry and CV integration for two annulated diquat compounds that are similar to the transquat compounds of the present application, but in which the charged nitrogen atoms are positioned on the same side of the core structure.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising”, as used herein, will be understood to mean that the list following is non-exhaustive and may or may not include any other additional suitable items, for example one or more further feature(s), component(s) and/or ingredient(s) as appropriate.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

The term “and/or” as used in a phrase such as “A and/or B” herein is intended to include “A and B”, “A or B”, “A”, and “B”.

The term “alkoxy”, as used herein, refers to straight-chain or branched alkyl group bonded to an oxygen. In some embodiments, the alkoxy group includes an alkyl having 1 to about 12 carbons, or 1 to about 8 carbons, or 1 to 6 carbons. Examples include, but are not limited to, methoxy, ethoxy, propoxy, butoxy, pentoxy, hexoxy, heptoxy and octoxy. By way of example, the term “C₁-C₆-alkoxy” refers to an alkoxy having 1 to 6 carbon atoms, such as, but not limited to, methoxy, ethoxy, n-propoxy, 1-methylethoxy, n-butoxy, 1-methylpropoxy, 2-methylpropoxy and 1,1-dimethylethoxy. “Alkoxy” is intended to embrace all structural isomeric forms of an alkoxy group. For example, as used herein, propoxy encompasses both n-propoxy and isopropoxy, etc.

The term “alkyl”, as used herein, refers to a monovalent saturated hydrocarbon chain of 1 to about 12, or 1 to about 8 carbons, or 1 to about 6, carbon atoms in length, such as, but not limited to, methyl, ethyl, propyl and butyl. The alkyl group may be a straight-chain, a branched-chain or cyclic. By way of example, the term “C₁-C₆-alkyl” as used herein refers to a saturated straight-chain or branched hydrocarbon having 1 to 6 carbon atoms. “Alkyl” is intended to embrace all structural isomeric forms of an alkyl group. For example, as used herein, propyl encompasses both n-propyl and isopropyl; butyl encompasses n-butyl, sec-butyl, isobutyl and tert-butyl.

The term “alkenyl”, as used herein, refers to a monovalent hydrocarbon chain of 1 to about 12, or 1 to about 8, carbon atoms in length that contains at least one carbon-carbon double bond. The alkenyl group may be a straight-chain, a branched-chain or cyclic. By way of example, the term “C₁-C₄-alkenyl” as used herein refers to a straight-chain or branched hydrocarbon having 1 to 4 carbon atoms and containing at least one carbon-carbon double bond. “Alkenyl” is intended to embrace all structural isomeric forms of an alkenyl group.

The term “alkynyl”, as used herein, refers to a monovalent hydrocarbon chain of 1 to about 12, or 1 to about 8, carbon atoms in length that contains at least one carbon-carbon triple bond. The alkynyl group may be a straight-chain, a branched-chain or cyclic. By way of example, the term “C₁-C₄-alkynyl” as used herein refers to a straight-chain or branched hydrocarbon having 1 to 4 carbon atoms and containing at least one carbon-carbon triple bond. “Alkynyl” is intended to embrace all structural isomeric forms of an alkynyl group.

The term “redox active material”, as used herein, refers to materials which undergo a change in oxidation state during operation of an electrochemical system, such as a flow battery. In certain embodiments, types of active materials comprise species dissolved in a liquid electrolyte. A type of redox active material may comprise a single species or may comprise multiple species.

Unless otherwise specified, each instance of an alkyl or alkoxy group is independently unsubstituted (an “unsubstituted alkyl”) or substituted (a “substituted alkyl”) with one or more substituents.

In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. Compounds described herein contemplates any and all such combinations in order to arrive at a stable compound. Heteroatoms such as nitrogen may have hydrogen substituents and/or any suitable substituent as described herein which satisfy the valencies of the heteroatoms and results in the formation of a stable moiety. Compounds described herein are not intended to be limited in any manner by the exemplary substituents described herein.

Exemplary substituents may include, for example, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

Substituents may themselves be substituted. For instance, the substituents of a “substituted alkyl” may include both substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls may be further substituted with alkyls, alkenyls, alkoxys, alkylthios, aminoalkyls, carbonyl-substituted alkyls, —CF₃, —CN, and the like.

The term “viologen” as used herein refers to a compound that includes a 4,4′-bypyridyl core structure. An example includes, but is not limited to, methyl viologen.

The present inventors have surprisingly found a class of viologen derivatives, referred to herein as transquat compounds and related species, that possess advantages when used as a redox active material in a battery, e.g., a redox flow battery. In particular, the transquat compounds provided herein exhibit high redox potential in comparison to compounds previously used as redox active materials in RFBs. These compounds are water-soluble and exhibit good chemical stability, both of which are important for effective use in RFBs. In some embodiments, the transquat compounds of the present application have a water-solubility of at least 0.05 mol/L, or at least 0.1 mol/L, or at least 0.5 mol/L, or at least 1 mol/L. In some embodiments, the water solubility of these compounds is as high as about 3 mol/L. The water-solubility is largely dependent on the substituents present on the transquat core structure.

In terms of stability, the present inventors have found that the transquat compounds can be stored as in aqueous solution under ambient conditions for over a month without detectably impurity formation (as determined by NMR).

Furthermore, the present transquat compounds exhibit high capacity retention. For example, the present transquat compounds can undergo redox cycling at least 90 times with negligible loss of capacity. Thus, the present application further provides the use of the transquat compounds in a high efficiency, long cycle life redox flow battery. Redox cycling of the present transquat compounds occurs rapidly and reversibly and provides high current density, high efficiency, and long lifetime in a flow battery.

The present application provides transquat compounds of Formula I

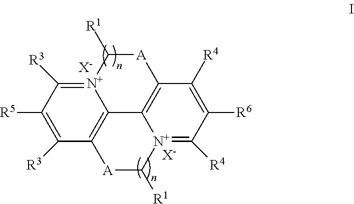

wherein:
   each A is CHR², NR², O, or SOₘ, where m is 0, 1 or 2;
   each R¹ and R² is independently H, optionally substituted C₁-C₆ alkyl, or optionally substituted C₁-C₆ alkoxy, optionally substituted di-alkylamino, optionally substituted di-arylamino, optionally substituted alkyl-arylamino, or a glycol side chain (such as ethylene glycol, propylene glycol, poly(ethylene glycol, or poly(propylene glycol);
   each R³, R⁴, R⁵, and R⁶ is independently H, optionally substituted C₁-C₆ alkyl, optionally substituted C₁-C₆ alkoxy, optionally substituted di-alkylamino, optionally substituted di-arylamino, optionally substituted alkyl-arylamino, or a glycol side chain (such as ethylene glycol, propylene glycol, poly(ethylene glycol, or poly(propylene glycol);
   each X is independently a counterion, such as a halide (e.g., Cl⁻ or Br⁻); and
   each n is independently 0, 1 or 2.

In accordance with some embodiments, there is provided a compound of Formula Ia

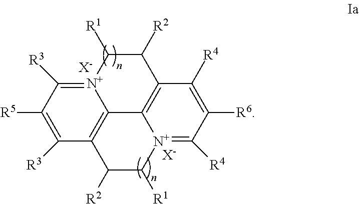

In some embodiments of the compound of Formula 1a, n is 1 or 2 and each R¹ is independently H, optionally substituted C₁-C₆ alkyl, or optionally substituted C₁-C₆ alkoxy. In some examples each R¹ is H.

In some embodiments, optionally in combination with the above embodiments, each R² is independently H, optionally substituted C₁-C₆ alkyl, or optionally substituted C₁-C₆ alkoxy. In some examples each R² is H.

In accordance with other embodiments, there is provided a compound of Formula Ib

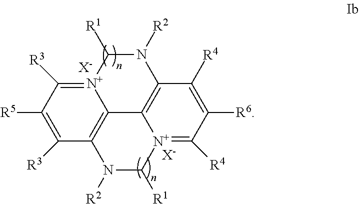

In some embodiments of the compound of Formula 1b, n is 1 or 2 and each R¹ is independently H, optionally substituted C₁-C₆ alkyl, or optionally substituted C₁-C₆ alkoxy. In some examples each R¹ is H.

In some embodiments, optionally in combination with the above embodiments, each R² is independently H, optionally substituted C₁-C₆ alkyl, or optionally substituted C₁-C₆ alkoxy. In some examples each R² is H.

In accordance with other embodiments, there is provided a compound of Formula Ic

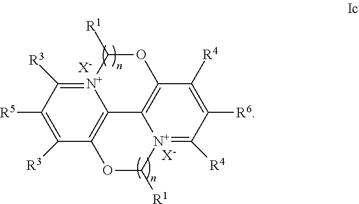

In some embodiments of the compound of Formula 1c, n is 1 or 2 and each R¹ is independently H, optionally substituted C₁-C₆ alkyl, or optionally substituted C₁-C₆ alkoxy. In some examples each R¹ is H.

In accordance with other embodiments, there is provided a compound of Formula Id

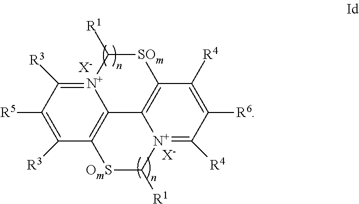

In some embodiments of the compound of Formula 1d, n is 1 or 2 and each R¹ is independently H, optionally substituted C₁-C₆ alkyl, or optionally substituted C₁-C₆ alkoxy. In some examples each R¹ is H.

In some embodiments of each of compounds Ia, Ib, Ic, and Id, as defined above, one or more of each R³ and R⁵ and/or one or more of each R⁴ and R⁶ is an alkyl, such as methyl, ethyl or propyl.

In some embodiments of each of compounds Ia, Ib, Ic, and Id, as defined above, one or more of each R³ and R⁵ and/or one or more of each R⁴ and R⁶ is an alkoxy, such as methoxy, ethoxy or propoxy.

Without wishing to be bound by theory, potential pathways of decomposition of an embodiment of the transquat compound of the present application are shown below. However, as noted herein, the inventors have found that these compounds are stable, even when stored in aqueous solution for over a month. Accordingly, such decomposition would only be minimal, if at all.

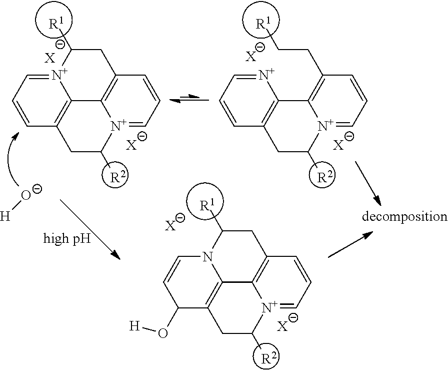

The compounds of Formula I undergo two reversible one-electron reductions, as illustrated in Scheme I below, using one embodiment of the transquat compounds of the present application for illustrative purposes only:

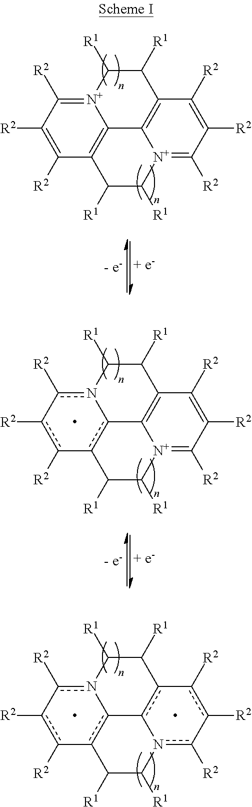

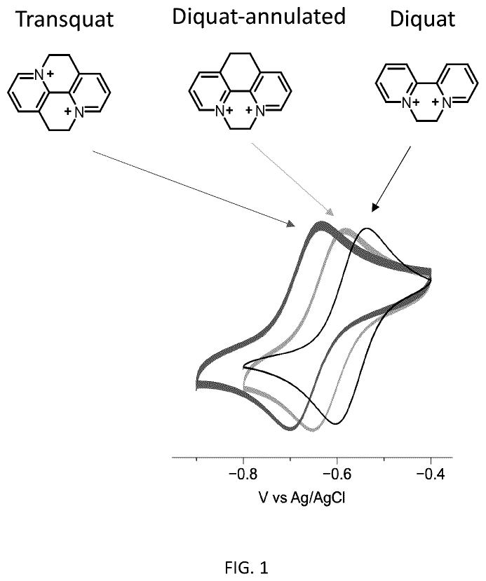

The present inventors have found that the first and second reductions of the transquat compounds are highly reversible. In addition, these compounds have been found to have a significantly more negative redox potential that diquats and annulated diquats, as illustrated in FIG. 1. The more negative redox potential of these compounds makes them particularly useful as electrolytes (negolytes) in RFBs.

Properties of the transquat compounds of the present application can be tuned, or optimized, for example for a particular application, by selection of the substituents. Incorporation of one or more electron-donating groups on one or both of the pyridinium rings can improve (i.e., lower) the redox potential of the negative electrolyte and increase overall cell voltage obtainable. This effect has been found with substituents on diquat compounds, where incorporation of the more strongly electron-donating group, methoxy, as a substituent on the pyridinium rings lowered the redox potential of the compound in comparison to substitution using methyl. The redox potential of the diquat compounds lowered as the strength of the substituent electon-donating effect increased (methoxy<methyl<pyridinyl<bromo groups). Following these findings this same effect has been found in the present transquat compounds, such that substituents that are strongly electron-donating will improve (i.e., lower) the redox potential of the transquat compounds.

Incorporation of large functional groups in one or more of the R³ and R⁴ substituents may reduce synthetic yields due to effects of steric hindrance. However, incorporation of such bulky substituents in these positions can also improve stability of the transquat compounds by preventing or minimizing attack by nucleophiles.

Alternatively, or in addition, incorporation of one or more polar or ionic group-containing substituents can further improve the solubility of the negative electrolyte.

Furthermore, selection of R¹ substituents is preferably made to exclude functional groups that promote dealkylation by stabilizing the resulting carbon cations.

By way of non-limiting example, the following transquat compounds include electron donating substituents on the pyridinium rings that have been incorporated to contribute to improved redox potential:

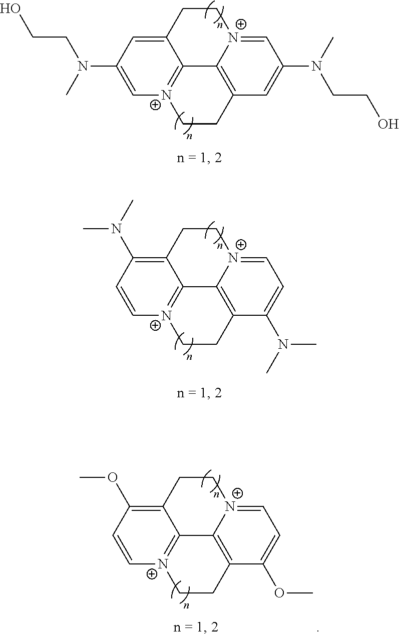

The following transquat compounds are non-limiting examples that include substitutions that can increase water solubility, thus energy density of flow batteries:

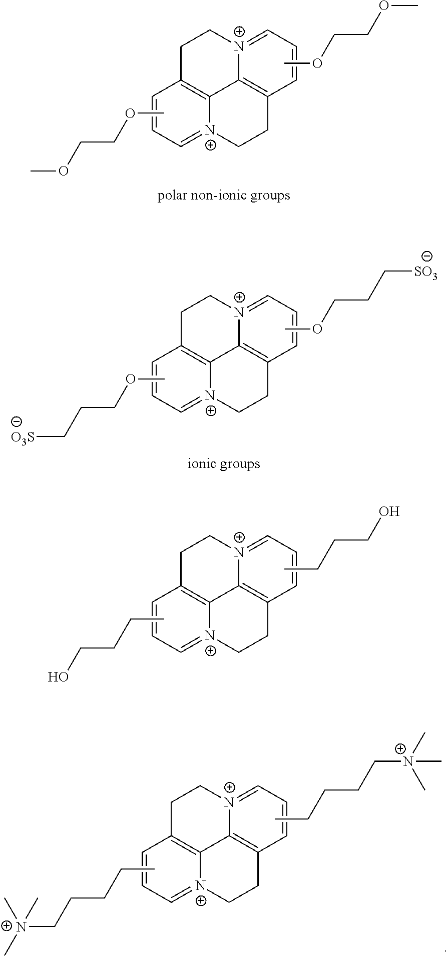

In some embodiments, the transquat compounds include substituents that function to increase both redox potential and water solubility. Non-limiting, illustrative examples of such compounds are provided below:

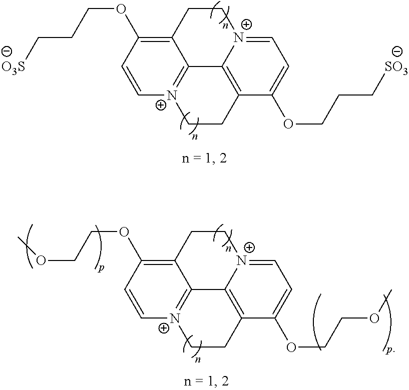

In this embodiment,

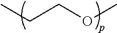

represents an ethyleneoxy moiety when p is 1 or a poly(ethylene oxide) moiety when p is greater than 1.

In addition to the effect of substituents on the transquat core structure, selection of the counterion can also influence the properties of the transquat compounds. This has been demonstrated using diquat compounds and confirmed using the present transquat compounds. For example, it has been found that bromide and chloride counterions can improve water solubility over iodide counterions. Other counterions that provide good water solubility include triflate and tosylate. In some instances, a combination of two different counterions can contribute to improved water solubility (e.g., bromide and chloride or triflate and chloride).

### Synthesis of Transquat Compounds

The transquat compounds of Formula I can be synthesized using a variety of methods. Two illustrative synthetic methods are detailed below.

### Five Step Synthesis

In one example of a synthetic process for manufacture of the transquat compounds of Formula I, precursors of the transquat compounds are synthesized using a method based on the procedure described in Ordronneau, L., et al. (3), an example of which is illustrated in Scheme 2.

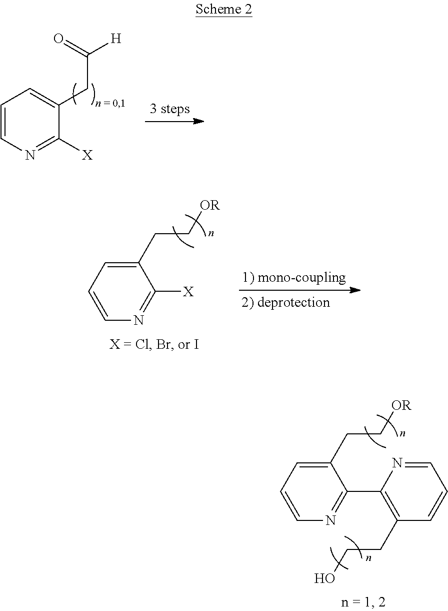

The resulting precursor compound is cyclized, for example as described below, to form the transquat compound of the present application.

This synthetic method is relatively expensive and, because of the number of steps, can suffer from low yields.

### Alternative Syntheses

In one embodiment there is provided an alternative, three-step, synthetic process for manufacture of the transquat compounds of Formula I. The first step of the process is functionalization of a bipyridine starting material, followed by a second oxidation step and then a final cyclization step.

As illustrated in Scheme 3, direct functionalization at the 3,3′-position of bipyridine, for the formation of a C2 bridge, can be achieved, for example, via an Rh-catalyzed C—H activation reaction as previously developed by Jung, Chang, and co-workers (4). Rhodium catalysts are known to be suitable promoters for activation of C—H bonds and include Rh(I), Rh(II) and Rh(III) catalysts.

The resultant functionalized compounds are then treated to convert the silyl groups to hydroxy groups, for example, using a Fleming-Tamao oxidation. Interestingly, the present inventors have found that the dimethoxy-methyl-silyl group oxidized faster than the methoxy-dimethyl-silyl group.

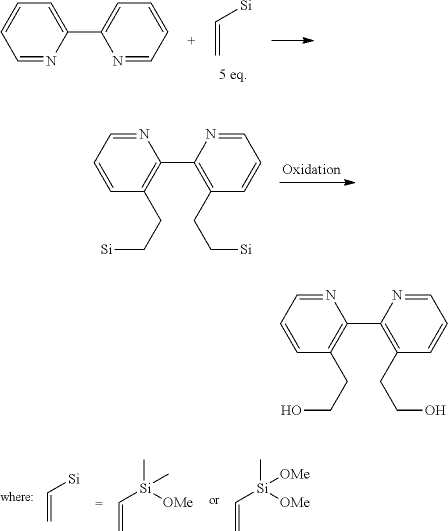

Alternatively, as illustrated in Scheme 4, direct functionalization at the 3,3′-position of bipyridine, for the formation of a C3 bridge, can be achieved, for example, via an Rh-catalyzed C—H activation reaction, using commercially available starting material. The 3,3′-functionalized compound is then oxidized to convert the silyl groups to hydroxy groups, for example, using a Fleming-Tamao oxidation.

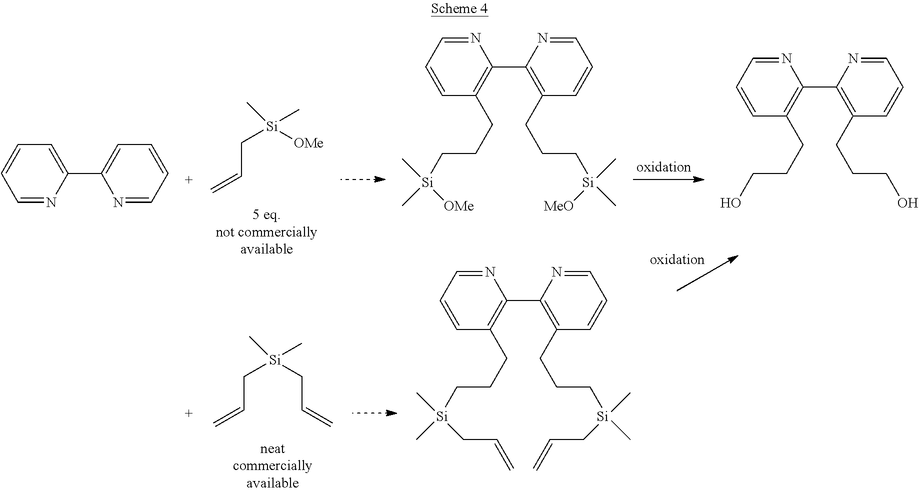

The final step of this synthesis is cyclization to form the transquat of Formula I. One example of the cyclization step comprises treatment of the product of the functionalization/oxidation steps with mesyl chloride and triethylamine, as illustrated in Scheme 5. The transquat product forms a solid that is readily isolated via solid-liquid separation (e.g., filtration). The salts remain in solution.

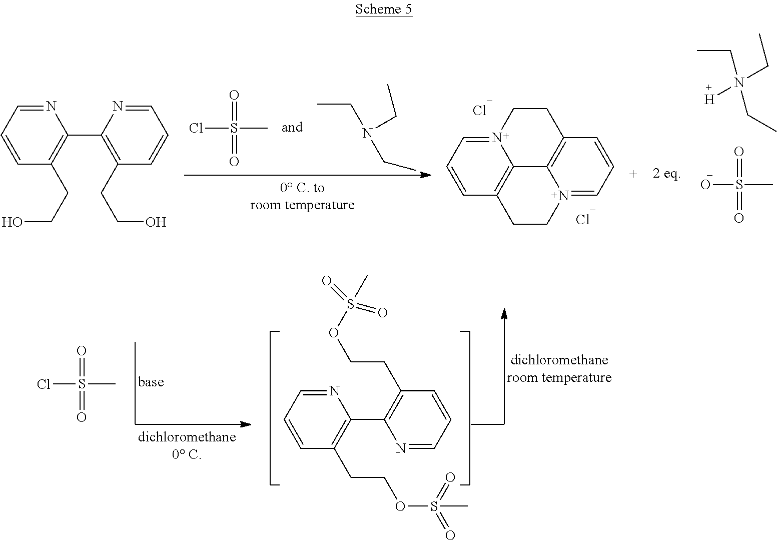

Exemplary transquat compounds prepared according to the process illustrated in Scheme 5 are provided in Table 1, and shown in relation to the corresponding bipyridine.

TABLE 1
Examples of transquat compounds

| Bipyridine | Transquat |
|---|---|
| 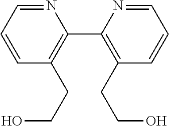 [SMILES: OCCc1cccnc1-c1ncccc1CCO] | 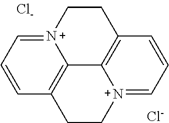 [SMILES: c1cc2c3[n+](c1)CCc1ccc[n+](c1-3)CC2 ; [Cl-]] |
| 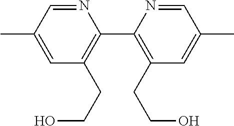 | 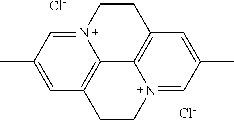 |
| 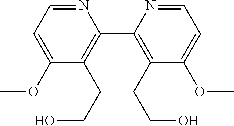 | 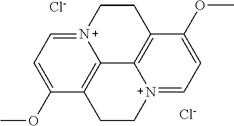 |
| 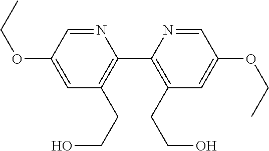 [SMILES: CCOc1cnc(-c2ncc(OCC)cc2CCO)c(CCO)c1] | 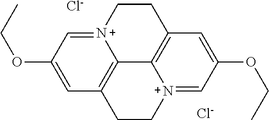 [SMILES: CCOc1cc2c3[n+](c1)CCc1cc(OCC)c[n+](c1-3)CC2 ; [Cl-] ; [Cl-]] |

Alternatively, the cyclization step can be performed by treatment of the product of the preceding functionalization/oxidation steps with thionyl chloride, followed by treatment with a strong acid. Examples of this cyclization are provided in Schemes 6 and 7. In one embodiment of this step, transquat compounds of Formula I can be synthesized by treatment of the intermediate bipyridine having alcohol sidechains with at least 2.5 equivalent SOCl₂ in a solvent, such as dichloromethane, with cooling. Upon warming to room temperature, the product transquat compound precipitates. Evaporation of all the solvent will also remove the HCl generated from the SOCl₂ treatment.

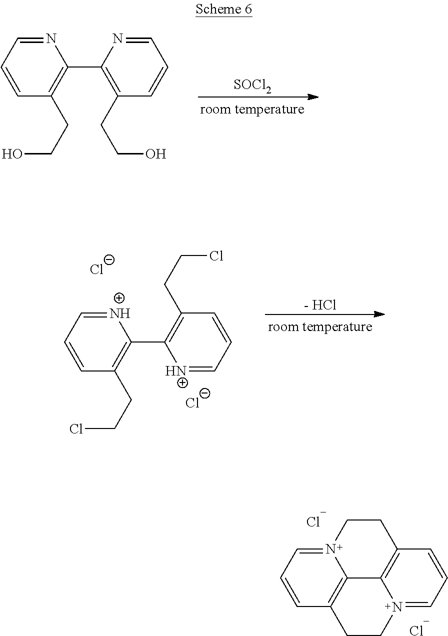

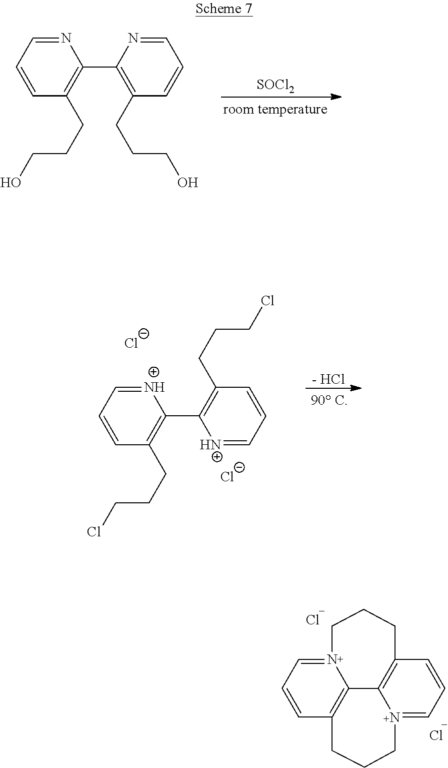

In the synthesis of compounds in which A is NR¹, O or SOₘ, the syntheses are analogous to the methods outlined above. The retrosynthetic routes are summarized below:

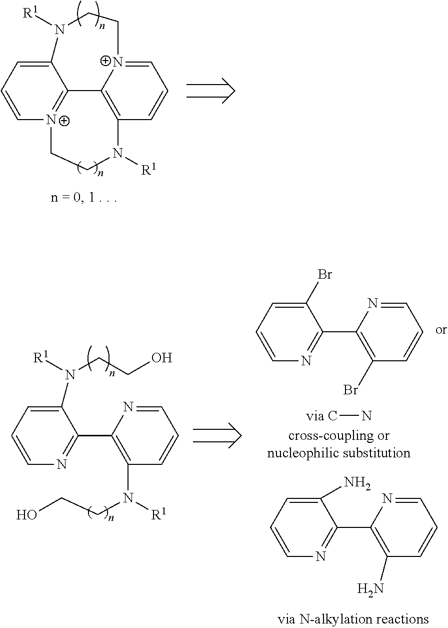

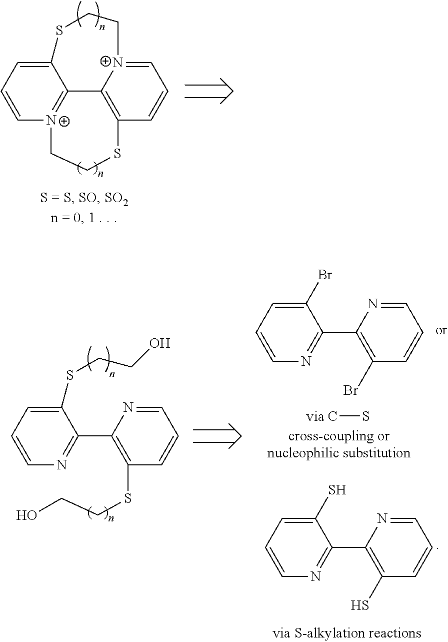

In another embodiment, there is provided an alternative synthetic process, which includes synthesis of the bipyridy intermediates with primary alcohol side chains via borylation followed by oxidation to form the C—OH bound, as illustrated in the scheme below:

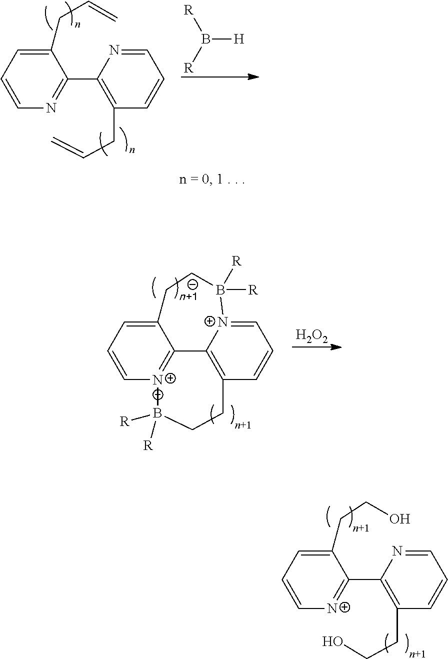

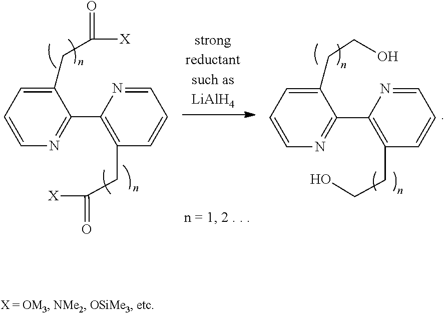

In another embodiment, there is provided an alternative synthetic process, which includes synthesis of the bipyridy intermediates with primary alcohol side chains via deprotonation followed by substitution, as shown in the scheme below:

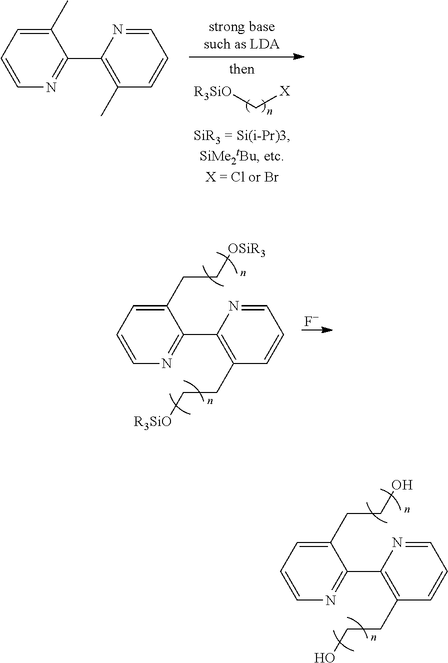

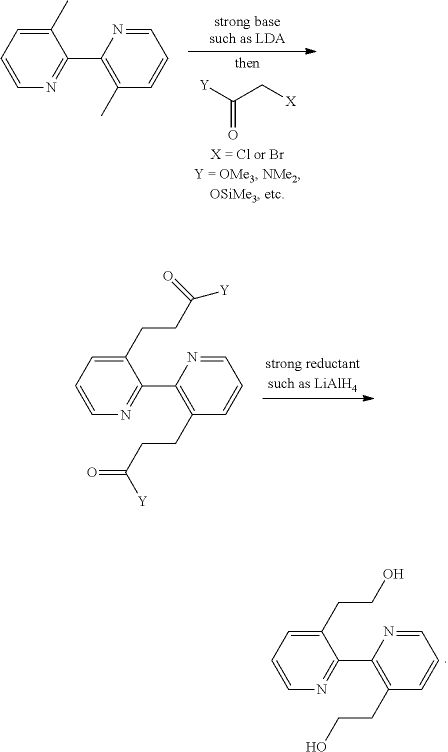

### Redox Flow Battery

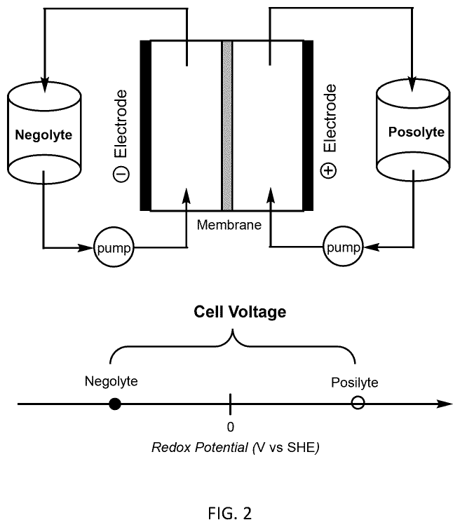

As briefly described above, RFBs are rechargeable batteries that store electrical energy in liquid solutions of redox active molecules. These solutions are stored in large tanks and circulated through cell stacks to charge or discharge electrical energy. An example of an RFB is illustrated in FIG. 2. RFBs are an attractive solution for storing grid-scale sustainable energy because their energy capacity scales easily with the size of electrolyte tank.

There are two major categories of RFB based on their electrolytes: inorganic and organic. Currently, only inorganic flow batteries such as vanadium, iron, and zinc-bromide systems have been demonstrated commercially. Organic RFBs (ORFBs) are divided into aqueous and non-aqueous systems; the former uses water as the cheaper, safer and greener solvent, while the latter uses organic solvents such as acetonitrile, which may offer much higher cell voltages.

The present application provides ORFBs, comprising one or more transquat compounds as described herein as an electrolyte, which store and release electric energy over repeated charging and discharging cycles.

Most of the key features of RFBs are tied to the chemical nature of their electrolytes. RFBs typically require two electrolytes: the posolyte (or catholyte) circulated through the positive electrode and the negolyte (or anolyte), which interacts with the negative electrode. During the discharging process, the posolyte is reduced to a lower degree of oxidation by accepting electrons from the positive electrode, while the negolyte is oxidized to a higher degree of oxidation by the negative electrode.

The aqueous organic redox flow batteries of the present application include a first redox active material and a second redox active material, at least one of which comprises one or more transquat compound of Formula I. The batteries may further include an aqueous electrolyte(s) (e.g., a first and second aqueous electrolyte), a separator, a first electrode, and a second electrode.

### Redox Active Materials

The present ORFBs include first and second redox active materials. The redox active materials may have one or more redox potentials. In certain embodiments, the redox potentials of the first redox active material and second redox active material may be the same or different. When the potentials are different the type of redox active material with the higher potential is the “positive redox active material”, and the corresponding electrolyte and electrode may be referred to as the “positive electrolyte” or posolyte and “positive electrode”. Likewise, the redox active material with the lower (more negative) potential is the “negative redox active material”, and the corresponding electrolyte and electrode may be referred to as the “negative electrolyte” or negolyte and “negative electrode”. During charge the positive redox active material present in the positive electrolyte undergoes oxidation, and the negative redox active material present in the negative electrolyte undergoes reduction, whereas during discharge, the positive redox active material present in the positive electrolyte undergoes reduction, and the negative redox active material present in the negative electrolyte undergoes oxidation.

Negative electrolytes previously used in ORFBs are typically large molecules classes, such as quinones, azo-aromatics, organic radicals and various dimers, trimers and polymers, each with their advantages and disadvantages. Viologens in general have extraordinary water solubility, in comparison to these large molecule classes, due to their di-cationic structures. Previously used molecule classes require further functionalization to reach similar level of solubilities and such functionalization can increase costs and complexity and can negatively impact redox potentials. Furthermore, the transquat compounds of the present application have a first reduction potential that is more negative than that of diquat and annulated diquat viologens. Accordingly, the transquat compounds of the present application are useful as the negative redox active material, or the negative electrolyte.

The positive electrolyte can include any suitable positive redox active material. The positive electrolyte can be selected from materials including:
a. transition-metal coordination compounds, including K₄[Fe(CN)₆] and substituted metallocenes, such as ferrocene methanol;
b. other organic materials, such as organic radicals, such as 4-Hydroxy-TEMPO, and their dimers, trimers, oligomers, and polymers; and
c. simple non-metallic inorganic species, such as Cl−/Cl₂, Br−/Br₂, I−/I₂, etc.

### Aqueous Electrolyte

The present ORFBs include an aqueous electrolyte(s). The redox active materials are present in the aqueous electrolyte(s). For example, the first redox active material may be present in a first aqueous electrolyte and the second redox active material may be present in a second aqueous electrolyte. The first and second aqueous electrolytes may be the same or different. In certain embodiments, the first aqueous electrolyte is the posolyte and the second aqueous electrolyte is the negolyte. In other embodiments, the first aqueous electrolyte is the negolyte and the second aqueous electrolyte is the posolyte.

The first and second redox active materials may be present in the first and second aqueous electrolytes, respectively, at a concentration of ≥1 M, ≥1.5 M, ≥2 M, ≥2.5 M, ≥3 M, ≥3.5 M, or >4 M. In certain embodiments, the first and second redox active materials may be present in the first and second aqueous electrolytes, respectively, at a concentration of from about 1M to about 10 M, such as from about 1M to about 8 M, from about 2M to about 6 M, or from about 3 M to about 5 M.

The first and second aqueous electrolytes may further include salts, acids, bases, supporting electrolytes, additives, co-solvents or combinations thereof.

### Separator

In accordance with most embodiments, the present ORFBs include a separator. The separator may be a porous separator. The separator may not be permeable to the transquat compounds (including transquat compounds in a salt form). In certain embodiments, the term separator is synonymous with membrane. Separators can be classified as permeable, semi-permeable, or non-permeable. The degree of permeability is dependent on the size of pores in the separator, the character (e.g., charge, hydrophobicity) of the pores, and the character of the electrolyte or electrolyte component which is to be transported across the separator. A porous separator is considered permeable to all electrolyte components, though the degree of permeability may differ for different component species of the electrolyte (e.g., based on size). A semi-permeable separator typically is selectively permeable to certain materials (e.g., small cations, small anions, H₂O) while being substantially non-permeable to other materials (e.g., large molecules, neutral species, a type of redox active material). In certain embodiments, the separator is a non-porous separator permeable to ions.

The separator may be ion permeable. In certain embodiments, the separator is selectively permeable to permit the flux of cations with low resistance, and may be termed “cation permeable” or “cation conductive”. In certain embodiments, the separator is selectively permeable to permit the flux of anions with low resistance, and may be termed “anion permeable” or “anion conductive”. Accordingly, the separator may be cation permeable or anion permeable. An ion selective separator may comprise functional groups of opposite charge to the permitted ion, such that the charge of the functional group repels ions of like charge. In certain embodiments, the separator is a cation exchange membrane. In certain embodiments, the separator is an anion exchange membrane. In other embodiments, the separator is a sulfonate-containing fluoropolymer, such as NAFION®. In still other embodiments, the separator is a sulfonated poly (ether ether ketone), polysulfone, polyethyelene, polypropylene, ethylene-propylene copolymer, polyimide, or polyvinyldifluoride. In some embodiments, the separator is functionalized with ammonium, SO₃H, OH, COOH or a combination thereof.

### Electrodes

The disclosed AORFBs can include one or more electrodes. In certain embodiments, the disclosed AORFBs include a first electrode and a second electrode. The first and second electrode may be the same material, or they may be different materials. In certain embodiments, the electrodes may include a carbon felt, carbon mesh, carbon foam, carbon cloth, carbon paper, or carbon plate. The electrode or electrodes are optionally coated with a catalyst to improve the efficiency of charge transfer at the electrode, for example, to reduce the charging and/or discharging overpotential. The electrode or electrodes can be coated with a poison, such as lead, to reduce the efficiency of current transfer, for example to reduce the current density of the hydrogen evolution reaction.

### Properties of the Organic Redox Flow Batteries

The AORFBs of the present application, which comprise one or more transquat compound of Formula I as a negative electrolyte, have advantageous electrochemical properties resulting a least from the chemical and electrochemical stability and improved negative redox potentials of these transquat compounds. These characteristics are useful for production long cycling AORFBs with improved energy storage capacity, compared to previously known AORFBs, and that do not require extreme conditions (as in, e.g., vanadium-based RFBs) to function.

### Further Applications

The transquat compounds of the present application are not limited to use in AORFBs. Rather these compounds can be used in other applications in which viologens are typically used. Examples of such applications and uses are provided in Ding, J. and co-workers' review article. (1)

Further, as should be readily apparent, the properties of the transquat compounds of Formula I, for example, in terms of the high reversibility of the first reduction, the low reduction potential, and the colour change between the reduced and oxidized forms, make these compounds useful in many applications beyond their use in energy storage (in AORFBs). Accordingly, the present application further provides methods of using these compounds as components of electrochromic materials, molecular machines, memory devices, gas storage and separation systems (as metal-organic framework, aka MOF), field-effect transistors, supercapacitors, redox-shuttles in electrochemistry, and materials in solar cells. Also provided herein are systems and devices that comprise one or more transquat compound of Formula I. Such systems and devices include, but are not limited to, electrochromic materials, molecular machines, memory devices, gas storage and separation systems (as MOF), field-effect transistors, supercapacitors, redox-shuttles, and solar cells.

To gain a better understanding of the invention described herein, the following examples are set forth. It should be understood that these examples are for illustrative purposes only. Therefore, they should not limit the scope of this invention in any way.

### EXAMPLES

### Example 1: Synthesis of Transquat Compounds

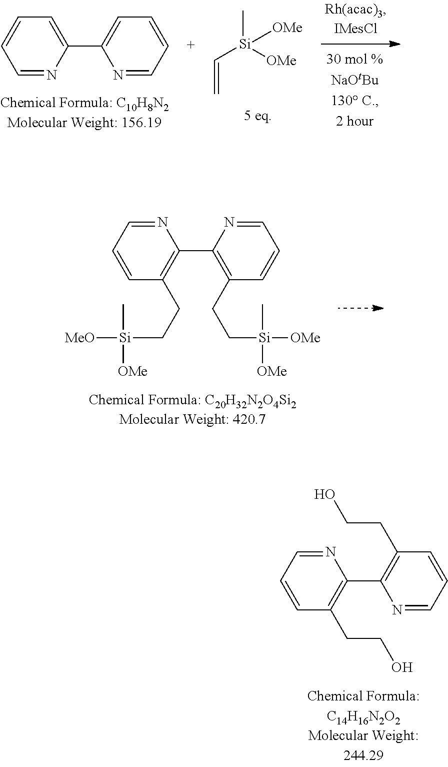

Step 1: An oven-dried 20 mL vial was added 2,2′-bipyridine (470 mg, 3.0 mmol), 1,3-bis(2,4,6-trimethylphenyl)imidazolium chloride (31 mg, 3 mol %), Rh(acac)₃ (36 mg, 3 mol %), sodium tert-butoxide (86 mg, 30 mol %) and toluene (5 ml). Dimethoxymethylvinylsilane (1.98 g, 15 mmol) was added and the mixture was purged with N₂ for 1 min. The mixture was vigorously stirred at 130° C. for 2 hours. After cooling, the solvent and excess reactants were removed under a vacuum. The crude mixture was purified by flash column chromatography (n-hexane/acetone, 20:1 to 1:1).

Step 2: The isolated intermediate (841 mg, 2.0 mmol) was dissolved in MeOH-THF (1:1) at room temperature, and KHCO₃ (880 mg, 10 mmol, 5 eq), KF (580 mg, 10 mmol, 5 eq), and H₂O₂ (˜680 mg, 20 mmol, 10 eq) were added. The mixture was heated at 60° C. overnight. Solvents were removed under a vacuum, 5 mL brine solution was added and the product was extracted with dichloromethane 5 mL three times (filtration may be needed due to the formation of silicates). The organic phase was dried over MgSO₄ and filtered. Solvents were removed from the filtrate to afford the crude mixture, which was purified by flash column chromatography (ethyl acetate/isopropanol, 20:1 to 1:1). The final product was 415 mg, yield 85%. Cyclization of the product resulted in formation of the transquat compound.

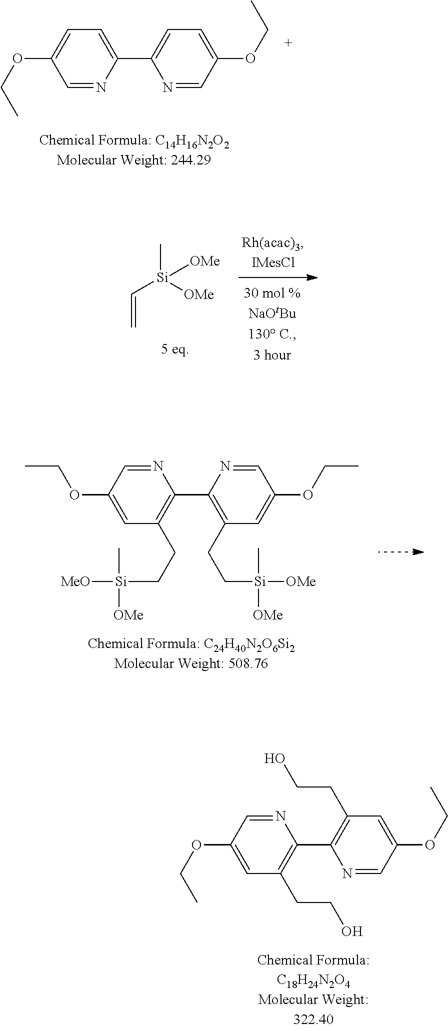

Step 1: A similar procedure was followed as described above, except 5,5′-diethoxy-2,2′-bipyridine (367 mg, 1.5 mmol), IMes chloride (15 mg, 3 mol %), Rh(acac)₃ (18 mg, 3 mol %), sodium tert-butoxide (43 mg, 30 mol %), dimethoxymethylvinylsilane (0.99 g, 7.5 mmol) were used. 10 ml of toluene was used due to the low solubility of the starting material. The mixture was vigorously stirred at 130° C. for 3 hours. The crude product was purified by flash with the same gradient. The yield of the intermediate was 580 mg (76% yield).

Step 2: 230 mg of the intermediate was oxidized using the preceding procedure with the same molar ratio of reagents affording 104 mg of the transquat product, yield 69%. Cyclization of the product resulted in formation of the transquat compound.

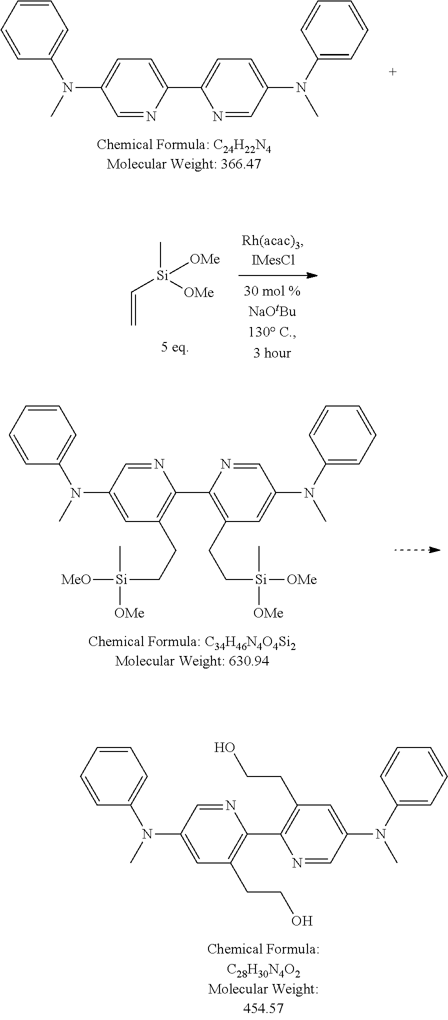

Step 1: A similar procedure was followed as described above, except 5,5′-bis(N-methyl-N-phenylamino)-2,2′-bipyridine (422 mg, 1.15 mmol), IMes chloride (15 mg, 3 mol %), Rh(acac)₃ (18 mg, 3 mol %), sodium tert-butoxide (43 mg, 30 mol %) dimethoxymethylvinylsilane (0.99 g, 7.5 mmol) and toluene (5 mL) was used. The mixture was vigorously stirred at 130° C. for 3 hours. The crude product was purified by flash column chromatography (n-hexane/acetone, 20:1 to 1:1). Yield of the intermediate was 620 mg (66% yield).

Step 2: 600 mg of the intermediate was oxidized using the preceding procedure with the same molar ratio of reagents affording 273 mg of the transquat product, yield 63%. Cyclization of the product resulted in formation of the transquat compound.

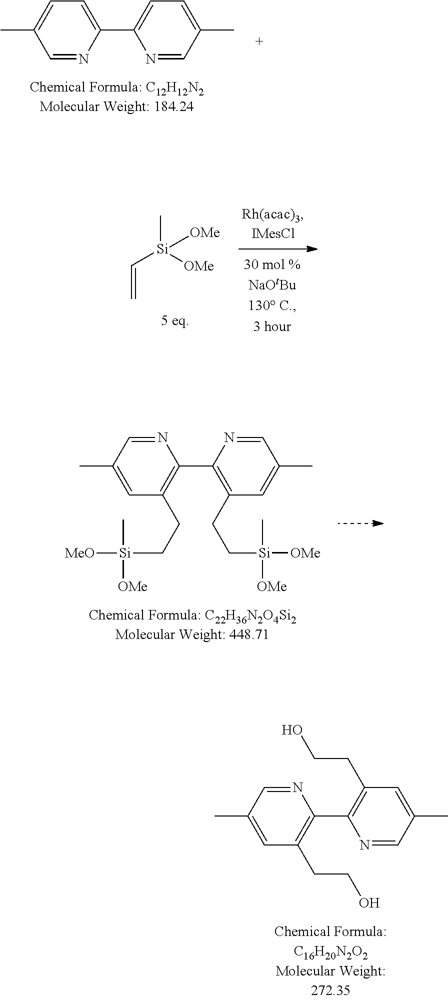

To an oven-dried 20 mL vial was added 5,5′-dimethyl-2,2′-bipyridine (276 mg, 1.5 mmol), 1,3-bis(2,4,6-trimethylphenyl)imidazolium chloride (15 mg, 3 mol %), Rh(acac)₃ (18 mg, 3 mol %), sodium tert-butoxide (43 mg, 30 mol %) and toluene (5 mL). Dimethoxymethylvinylsilane (0.99 g, 7.5 mmol) was added and the mixture was purged with N₂ for 1 min. The mixture was vigorously stirred at 130° C. for 3 hours. The crude product was purified by flash column chromatography (n-hexane/acetone, 20:1 to 1:1). Yield of the intermediate was 559 mg (yield 83%). The intermediate was converted into the final bipyridine product in 69% yield. Cyclization of the bipyridine product resulted in formation of the transquat compound.

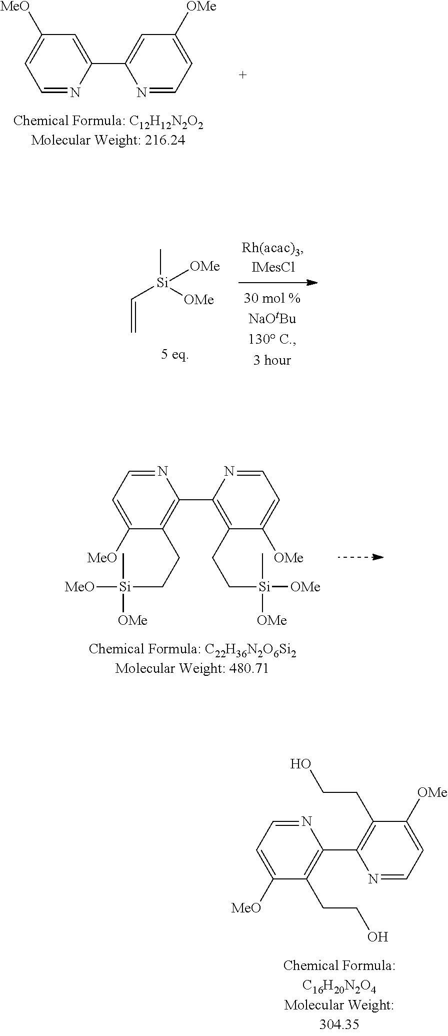

To an oven-dried 20 mL vial was added 4,4′-diethoxy-2,2′-bipyridine (324 mg, 1.5 mmol), 1,3-bis(2,4,6-trimethylphenyl)imidazolium chloride (15 mg, 3 mol %), Rh(acac)₃ (18 mg, 3 mol %), sodium tert-butoxide (43 mg, 30 mol %) and toluene (5 mL). Dimethoxymethylvinylsilane (0.99 g, 7.5 mmol) was added and the mixture was purged with N₂ for 1 min. The mixture was vigorously stirred at 130° C. for 3 hours. The crude product was purified by flash column chromatography (n-hexane/acetone, 20:1 to 1:1). Yield of the intermediate was 375 mg (yield 52%). The intermediate was converted into the final bipyridine product in 61% yield. Cyclization of the bipyridine product resulted in formation of the transquat compound.

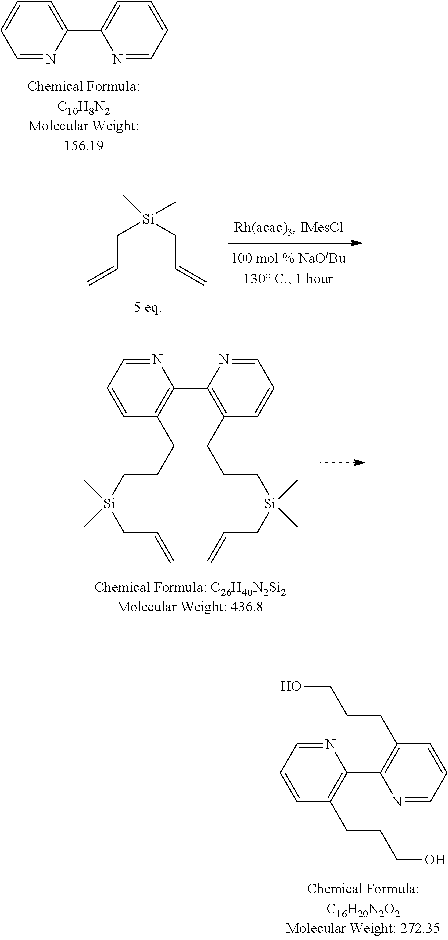

STEP 1: To an oven-dried 20 mL vial was added 2,2′-bipyridine (470 mg, 3.0 mmol), 1,3-bis(2,4,6-trimethylphenyl)imidazolium chloride (31 mg, 3 mol %), Rh(acac)₃ (36 mg, 3 mol %), sodium tert-butoxide (287 mg, 100 mol %) and toluene (1 mL). Diallyldimethylsilane (3.15 g, 22.5 mmol) was added and the mixture was purged with N₂ for 1 min. The mixture was vigorously stirred at 130° C. for 4 hours. The mixture was diluted with 10 mL 1:10 (v/v) mixture of ethyl acetate and hexanes, filtered through a pad of celite to remove most of the catalyst and salts, and concentrated under vacuum to afford the crude mixture. The crude product was purified by flash column chromatography (n-hexane/acetone, 20:1 to 1:1). The silyl-bipyridine intermediate was obtained as a colorless liquid (699 mg, 53% isolated yield), and a more polar cyclic side-product was also isolated (257 mg, 29% isolated yield).

STEP 2: Next, the silyl-bipyridine intermediate (437 mg, 1.0 mmol) was dissolved in 5 mL of CHCl₃. KHF₂ (469 mg, 6 mmol) and CF₃COOH (1.37 g, 12 mmol) were added sequentially at room temperature, and the mixture was heated at 50° C. for 3 h, with the lid slightly open to release pressure. After the evaporation of volatile materials, the remaining liquid was dissolved in 5 mL of a 1:1 (v/v) mixture of THF and methanol. NaHCO₃ (1.26 g, 15 mmol) and 30% H₂O₂ (ca. 4.2 ml; 40 mmol) were slowly added to the mixture which was then heated at 70° C. overnight. [septum cap with needle] The precipitated silicates were filtered off, after which solvents were removed under vacuum. Brine solution of 5 mL was added and the product was extracted with dichloromethane 5 mL three times. The crude product was purified by flash column chromatography (ethyl acetate/2-propanol, v/v, 19:1 to 1:1). The final product as a colorless viscous liquid that slowly crystallizes at room temperature, 231 mg was obtained (77% yield). Cyclization of the product resulted in formation of the transquat compound.

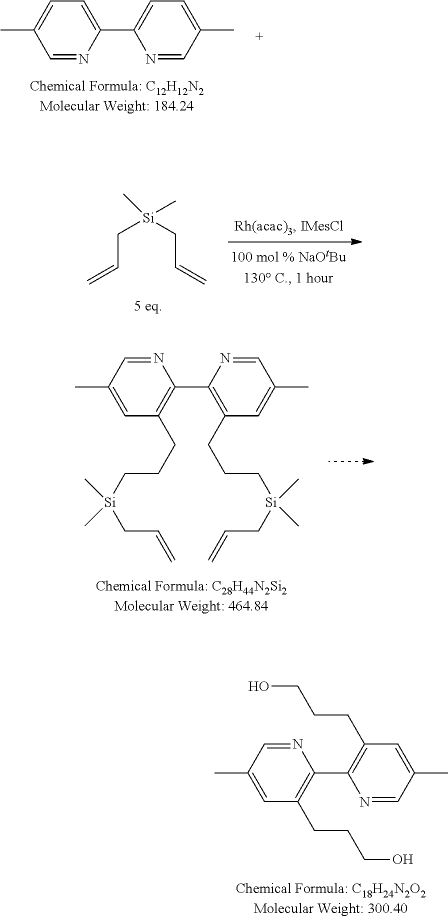

Product was obtained following a similar procedure as above, except that 5,5′-dimethyl-2,2′-bipyridine (552 mg, 3.0 mmol) was used. The silyl-bipyridine intermediate was obtained as a colorless liquid (871 mg, 62% isolated yield), and a more polar cyclic side-product was also isolated (196 mg, 20% isolated yield). 405 mg of the major product was oxidized into diol, yielding 198 mg final product as a white solid (76% yield). Cyclization of the product resulted in formation of the transquat compound.

### 5,6,11,12-tetrahydrodipyrido[3,2,1-de:3′,2′,1′-ij][1,5]napyridine-4,10-diium

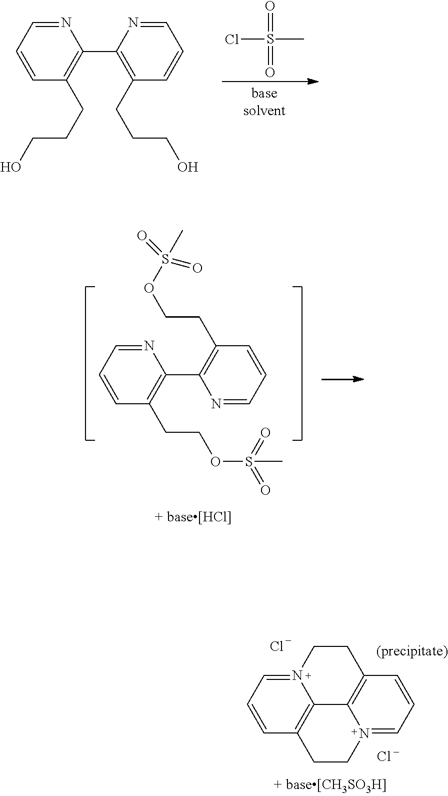

This compound was prepared from 2,2′-([2,2′-bipyridine]-3,3′-diyl)bis(ethan-1-ol), which was synthesized using Rh-catalyzed C—H activation reactions as described above. The starting diol (1 mmol) was dissolved in a non-protic organic solvent, such as dichloromethane or chloroform, to which was added 2.2 mmol of the base, such as triethylamine or diisopropylamine. With cooling and vigorous stirring, 2.1 equivalent of methane sulfonyl chloride was added, and the reaction was stirred for 1 to 12 hours at a temperature between 25 to 80° C. The reaction was filtered to collect the precipitate, which was washed extensively with an appropriate solvent, such as hexanes, diethyl ether or dichloromethane, to remove the salt of the protonated base. The product can be further purified by recrystallization in 2-propanol, methanol or their mixtures with water. 1H NMR (500 MHz, D2O) δ 8.95 (dt, J=6.1, 0.6 Hz, 1H), 8.71-8.48 (m, 1H), 8.13 (dd, J=8.1, 6.0 Hz, 1H), 5.10-4.84 (t, J=6.9 Hz, 3H), 3.59 (t, J=6.9 Hz, 3H). 13C NMR (126 MHz, D2O) δ 146.68, 146.05, 135.99, 129.91, 53.47, 24.55.

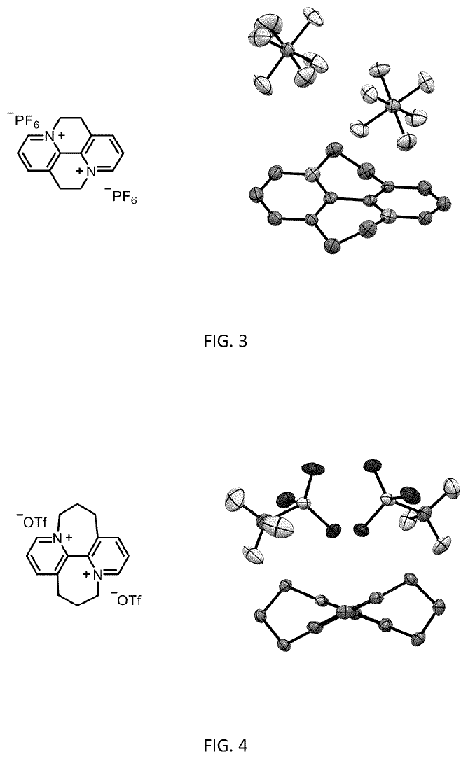

The structure of the hexafluorophosphate salt of this transquat compound has been resolved using X-ray crystallography, as shown in FIG. 3.

### 4,5,6,10,11,12-hexahydro-3a,9a-diazadibenzo[ef,kl]heptalene-3a,9a-diium dichloride

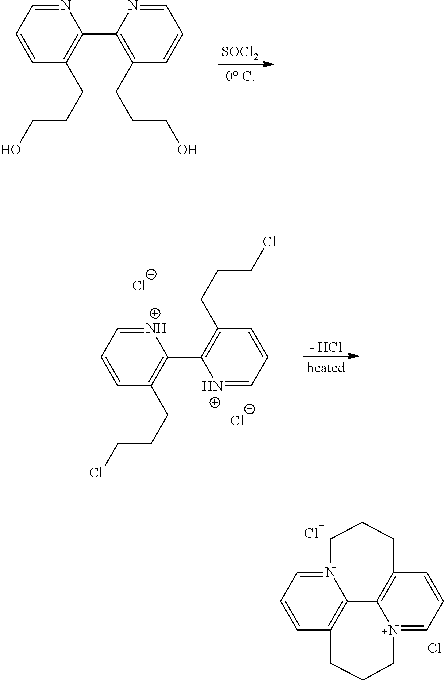

The transquat compound was prepared from bpy-C3—OH, synthesized as described above, which was treated with at least 2.5 equivalent SOCl₂ in dichloroethane or acetonitrile with cooling. Upon warming to room temperature, no precipitates formed until the solution was heated to above 85° C. The desirable precipitate slowly formed as a white solid. Evaporation of all the solvents yielded crude product which was purified by crystallization in methanol (or 2-isopropanol) and diethyl ether. ₁H NMR (500 MHz, D₂O) δ 9.05 (dd, J=6.0, 1.3 Hz, 2H), 8.63 (dd, J=8.2, 1.3 Hz, 2H), 8.19 (dd, J=8.2, 6.1 Hz, 2H), 4.82-4.78 (t, m, 2H), 4.42-4.36 (t, m, 2H), 3.16-1.22 (t, m, 2H), 2.76-2.68 (t, m, 2H), 2.58-2.51 (t, m, 2H), 2.48-2.41 (t, m, 2H). ¹³C {¹H} NMR (126 MHz, D₂O) δ 146.88, 141.86, 130.88, 119.58, 57.98, 31.24, 26.62.

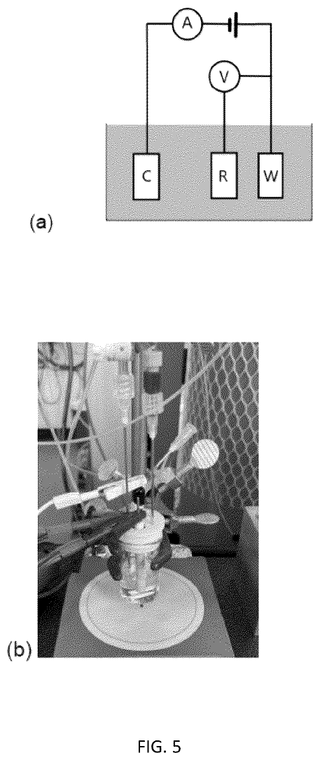

The structure of the triflate salt of this transquat compound has been resolved using X-ray crystallography, as shown in FIG. 4.

### Example 2: Electrochemistry and Water Solubility of Transquat Compounds

### Electrochemistry

The electrochemical properties and performances of transquat compounds of the present application were determined using two distinct techniques. Cyclic voltammetry (CV) and differential pulse voltammetry (DPV) were used to elucidate important electrochemical characteristics and test the prepared molecules in a standard three electrode cell setup.

The three-electrode setup consists of a working electrode, which is the active electrode on which redox reactions are occurring, a counter electrode (or auxiliary electrode), which provides a circuit and establishes a means to apply or measure electrical current; and a reference electrode, an electrode with a well-known and pre-defined electric potential, against which the measured electric potential is defined. All three electrodes are then submerged in a solution containing the dissolved electrolytes in a closed cell. A diagram of the three-electrode setup used in these tests is shown below in FIG. 5a. In practice, the closed cell typically consists of a glass vessel with an engineered cap, pre-made holes for the three electrodes, and nitrogen lines if the absence of oxygen is necessary for experiments. A sample photograph of the three-electrode setup used in these electrochemical measurements is shown below in FIG. 5b.

Both electrochemical measurement techniques used in this Example are techniques of voltammetry, which measure the current of a given analyte as a function of the applied electric potential, defined in reference to the chosen reference electrode. The standard hydrogen electrode (SHE) is defined by default as having a potential of 0.000 V, and the potentials of all other reference electrodes are defined as potential differences from the SHE. The voltammetry experiments in this work utilize the Ag/AgCl reference electrode in 3 M NaCl solution, which has a defined potential of +0.200 V from the SHE. As such, the redox potentials measured in these experiments by CV or DPV will have an offset of +0.200 V applied to report the redox potential with reference to the SHE accurately. In these experiments, a platinum wire was used as the counter electrode. A planar glassy carbon electrode was used as the working electrode to provide an overpotential of the HER of water.

### Pulse Voltammetry Measurements

Pulse voltammetry techniques such as Square Wave Voltammetry (SWV) and Differential Pulsed Voltammetry (DPV) were employed to identify the first reduction event of transquats. These techniques are known to have suppressed non-Faradaic currents and, therefore, a relatively flat baseline on Current/Voltage plots which is beneficial for peak-finding.

### Cyclic Voltammetry Scans

Cyclic voltammetry (CV) was employed to evaluate the stability of transquats under repeated charging-discharging cycles. The peak center was found by pulsed techniques as above. In contrast, the scanning range was about 300 mV above the oxidation peak and about 300 mV below the reduction peak or the center between this peak and the 2nd reduction peak, whichever is more positive in potential. The integrated area under the curves over 100 scans can be used to preliminarily evaluate the redox stability of transquats on carbon electrodes.

### Water Solubility

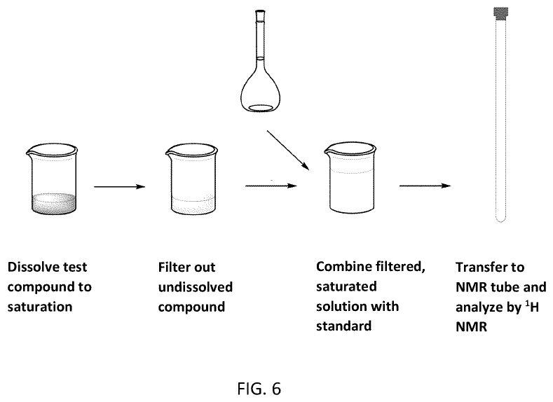

The solubility of the transquat compounds was determined using quantitative NMR according to the method summarized in FIG. 6. The compound to be tested was dissolved in 100 μl D₂O until saturation was reached. Any undissolved compound was filtered off and the filtrate was combined with a standard solution containing a known amount of a calibration compound as an internal reference. The sample is investigated using 1H NMR. The resultant one-dimensional NMR spectrum will have areas under peaks proportional to concentrations of the compound to be tested and the calibration compound. The solubility of the compound to be tested was then calculated from a relative concentration determination based on the known calibration compound concentration.

### Diffusion Coefficient

The diffusion coefficient of molecules was measured using Levic plot, obtained using a rotating disk electrode (RDE) setup according to standard procedures.

### Results

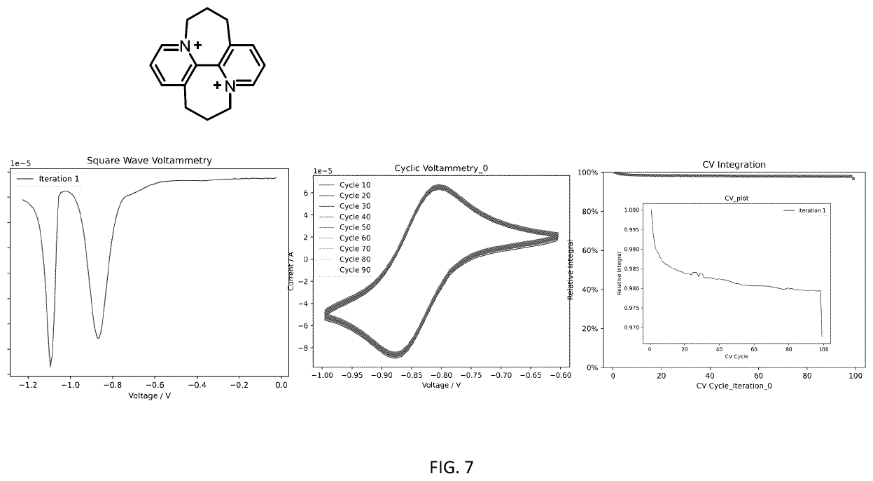

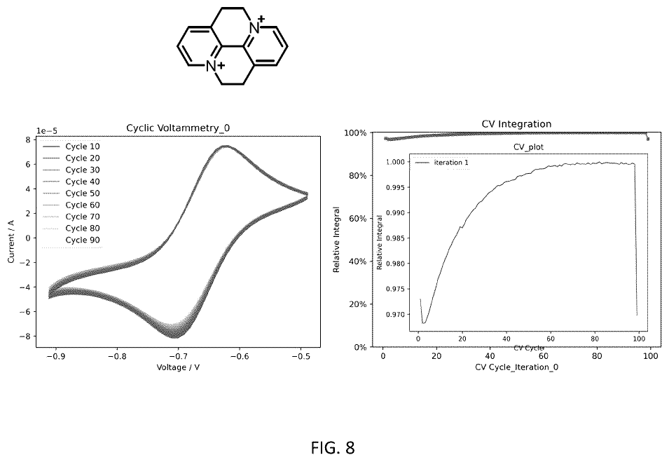

Pulse voltammetry and cyclic voltammetry scans for exemplary embodiments of the transquat compounds of the present application are provided in FIGS. 7 and 8. Results from testing exemplary transquat compounds are summarized in Table 2:

TABLE 2
Electrochemical and Solubility Properties of Transquat Compounds

| | Redox | Capacity | Solubility in | Reaction | Diffusion |
|---|---|---|---|---|---|
| | Potential (V | over 100 | water (M, at | Rate (k°, in | coefficient |
| | vs SHE) | cycles | 20°C) | cm/s) | (D, in cm²s⁻¹) |
| 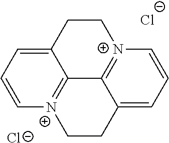 [SMILES: [Cl-] ; c1cc2c3[n+](c1)CCc1ccc[n+](c1-3)CC2 ; [Cl-]] | −0.48 | 0.98 | >1.5 | 7.64 × 10⁻³ | 1.62 × 10⁻⁵ |
| 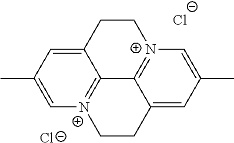 | −0.59 | 0.95 | ~1 | 1.60 × 10⁻² | 6.45 × 10⁻⁶ |
| 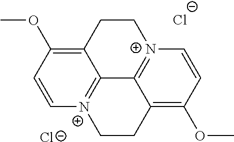 | −0.80 | 0.80 | Low | 4.14 × 10⁻³ | 2.5 × 10⁻⁶ |
| 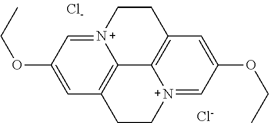 [SMILES: CCOc1cc2c3[n+](c1)CCc1cc(OCC)c[n+](c1-3)CC2 ; [Cl-]] | −0.80 | 0.99 | >1.5 | 6.15 × 10⁻³ | 1.41 × 10⁻⁵ |
| 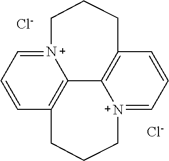 [SMILES: c1cc2c3[n+](c1)CCCc1ccc[n+](c1-3)CCC2 ; [Cl-] ; [Cl-]] | −0.64 | 0.98 | >1.5 | 1.94 × 10⁻² | 1.63 × 10⁻⁵ |

A comparison of the properties of a transquat compound according to the present application to properties of a similar diquat and annulated diquat compounds is provided in Table 3:

TABLE 3
Comparison of Electrochemical and Solubility Properties of Transquat, Diquat and
Annulated Diquat Compounds

| | Redox | Capacity | Solubility in | Reaction | Diffusion |
|---|---|---|---|---|---|
| | Potential | over 100 | water | Rate | coefficient |
| | (V vs SHE) | cycles | (M, at 20° C.) | (k°, in cm/s) | (D, in cm²s⁻¹) |
| 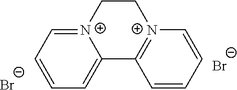 [SMILES: [Br-] ; c1cc[n+]2c(c1)-c1cccc[n+]1CC2 ; [Br-]] | −0.37 | 0.98 | >1.5 | N/A | N/A |
| 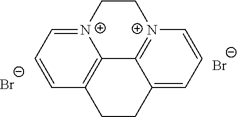 [SMILES: [Br-] ; c1cc2c3[n+](c1)CC[n+]1cccc(c1-3)CC2 ; [Br-]] | −0.42 | 0.98 | >1.5 | 7.68 × 10⁻³ | 9.87 × 10⁻⁶ |
| 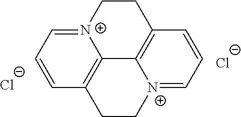 [SMILES: [Cl-] ; c1cc2c3[n+](c1)CCc1ccc[n+](c1-3)CC2 ; [Cl-]] | −0.48 | 0.98 | >1.5 | 7.64 × 10⁻³ | 1.62 × 10⁻⁵ |

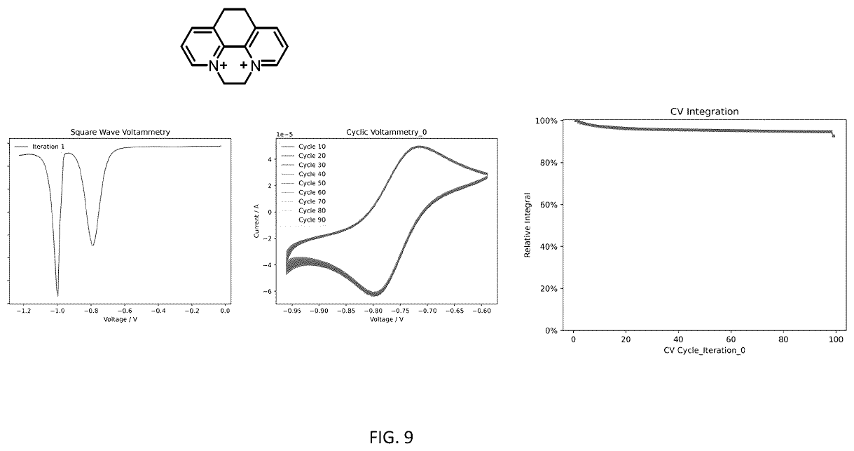

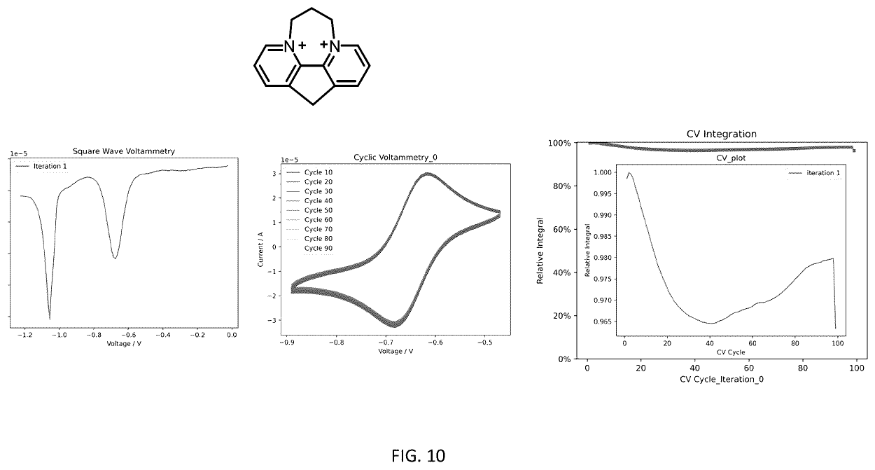

The results summarized above and shown in the Figures illustrate that the transquat compounds of the present application are highly reversible redox-active compounds having good stability following under repeated charging-discharging cycles. Furthermore, these compounds exhibit reduction peaks that are significantly more negative that alternative electrolyte compounds, such as diquats and annulated diquats (see FIG. 1 and Table 3 for direct comparison), and compounds similar to the present transquats but having charged nitrogen atoms on the same side of the core structure (rather than opposite sides; see FIGS. 9 and 10). Consequently, the transquat compounds of the present application are suitable for effective use in ORFBs and exhibit improved electrochemical properties and water solubility in comparison to alternative electrolyte compounds.

### REFERENCES

1) Ding, J. et al. “Viologen-inspired functional materials: synthetic strategies and applications.” J. Mater. Chem. A Mater. Energy Sustain. 2019, 7, 23337-23360.
2) Rubén Rubio-Presa, Lara Lubián, Mario Borlaf, Edgar Ventosa, and Roberto Sanz, “Addressing Practical Use of Viologen-Derivatives in Redox Flow Batteries through Molecular Engineering” ACS Materials Letters 2023 5 (3), 798-802.
3) Ordronneau, L., Carella, A., Pohanka, M., and Simonato, J-P., “Chromogenic detection of Sarin by discolouring decomplexation of a metal coordination.” Chem. Commun., 2013,49, 8946-8948.
4) Jaesung Kwak, Youhwa Ohk, Yousung Jung, and SukbokChang, “Rollover Cyclometalation Pathway in Rhodium Catalysis: Dramatic NHC Effects in the C—H Bond Functionalization.” J. Am. Chem. Soc. 2012, 134, 17778-17788.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A compound having the structure of Formula I 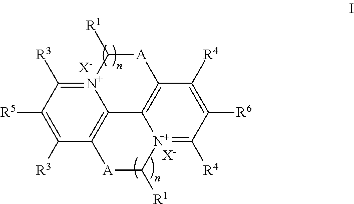
wherein:
each A is CHR², NR², O, or SOₘ, where m is 0, 1 or 2;
each R¹ and R² is independently H, optionally substituted C₁-C₆ alkyl, or optionally substituted C₁-C₆ alkoxy, optionally substituted di-alkylamino, optionally substituted di-arylamino, optionally substituted alkyl-arylamino, or a glycol side chain (such as ethylene glycol, propylene glycol, poly(ethylene glycol, or poly(propylene glycol);
each R³, R⁴, R⁵, and R⁶ is independently H, optionally substituted C₁-C₆ alkyl, or optionally substituted C₁-C₆ alkoxy, optionally substituted di-alkylamino, optionally substituted di-arylamino, optionally substituted alkyl-arylamino, or a glycol side chain (such as ethylene glycol, propylene glycol, poly(ethylene glycol, or poly(propylene glycol);
each X⁻ is independently a counterion, such as a halide (e.g., Cl⁻ or Br⁻), a triflate or a tosylate; and
each n is independently 0, 1 or 2.

2. The compound of claim 1 having the structure of Formula la, Ib, Ic, or Id

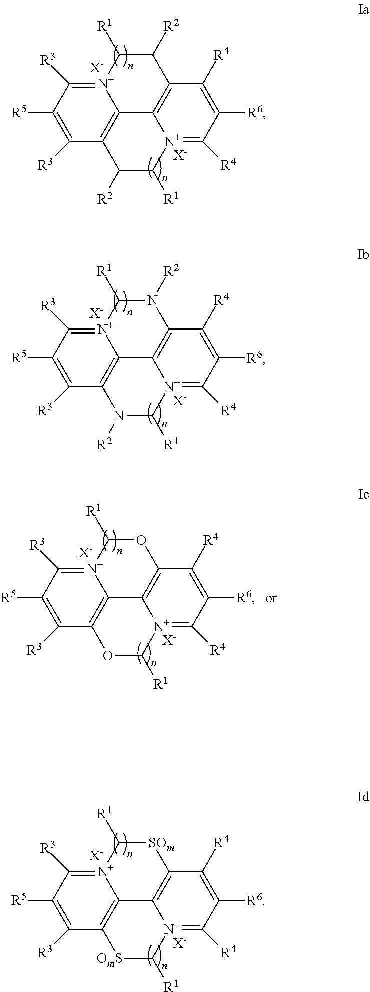

3. The compound of claim 2, wherein the compound has the structure of Formula Ia or Ib and each R² is independently H, optionally substituted C₁-C₆ alkyl, or optionally substituted C₁-C₆ alkoxy, or wherein each R² is H.

4. The compound of claim 1, wherein each R¹ is independently H, optionally substituted C₁-C₆ alkyl, or optionally substituted C₁-C₆ alkoxy, or wherein each R¹ is H.

5. The compound of claim 1, wherein one or more of each R³ and R⁵ and/or one or more of each R⁴ and R⁶ is an alkyl.

6. The compound of claim 5, wherein the alkyl is methyl, ethyl or propyl.

7. The compound of claim 1, wherein one or more of each R³ and R⁵ and/or one or more of each R⁴ and R⁶ is an alkoxy.

8. The compound of claim 7, wherein the alkoxy is methoxy, ethoxy or propoxy.

9. The compound of claim 1, which is a salt of:

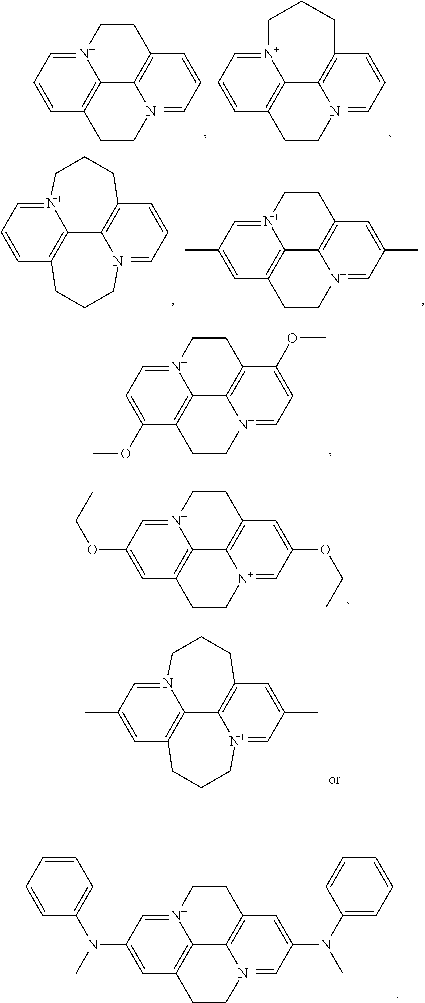

10. The compound of claim 1, which is a salt of: 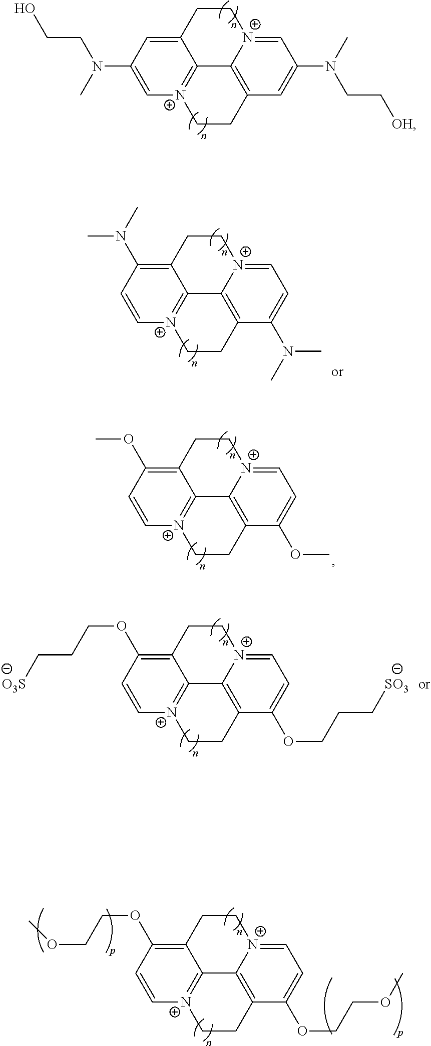
wherein each n is independently 1 or 2, and wherein 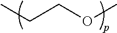
represents an ethyleneoxy moiety when p is 1 or a poly(ethylene glycol) moiety when p is greater than 1.

11. The compound of claim 1, which is a salt of:

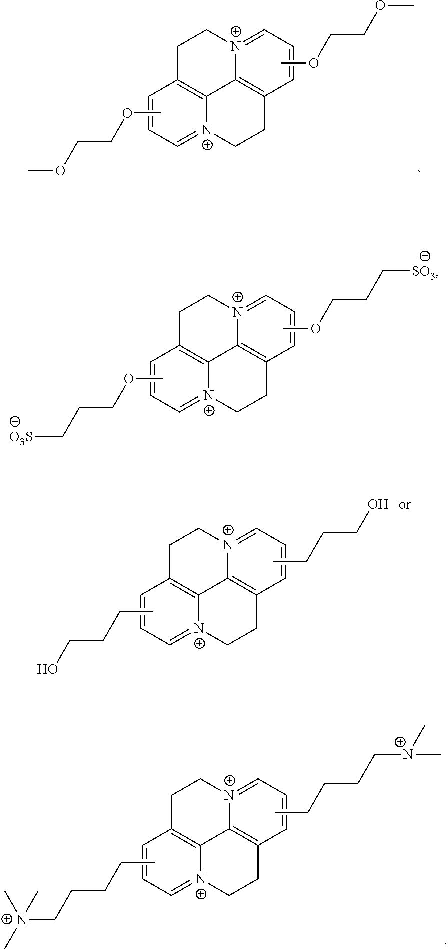

12. The compound of claim 1, wherein each X is independently Cl⁻ or Br⁻.

13. A redox flow battery comprising:
(a) a negative electrode;
(b) a first redox active composition comprising one or more compound according to claim 1 as a negative electrode electrolyte, the negative electrode electrolyte contacting the negative electrode;
(c) a positive electrode;
(d) a second redox active composition comprising a positive electrode electrolyte contacting the positive electrode; and
(e) an ion selective membrane interposed between the negative electrode and the positive electrode.

14. The redox flow battery according to claim 13, wherein the positive electrode electrolyte comprises (i) a transition-metal coordination compound, such as K₄[Fe(CN)₆] or a substituted metallocene, such as ferrocene methanol; (ii) an organic material, such as an organic radical or a dimer, trimer, oligomer, or polymer thereof; or (iii) a non-metallic inorganic material.

15. The redox flow battery according to claim 13, wherein each the one or more compound has the structure of Formula Ia, Ib, Ic, or Id

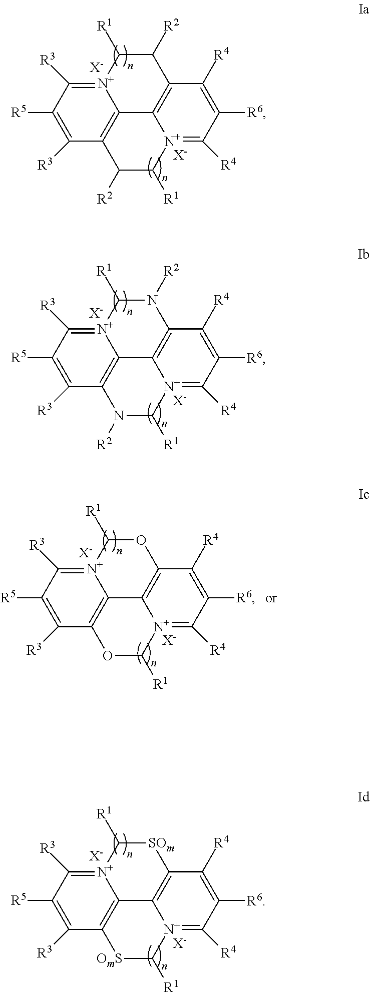

16. The redox flow battery according to claim 13, wherein the one or more compound is a salt of:

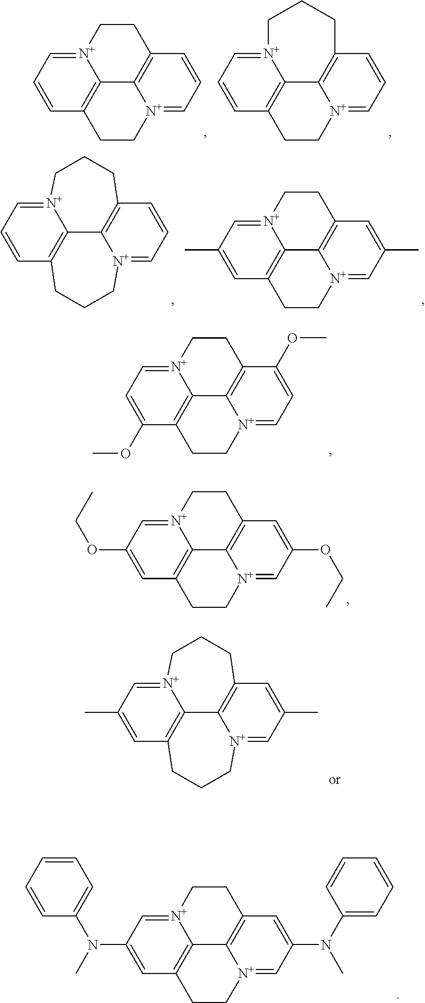

17. The redox flow battery according to claim 13, wherein the one or more compound is a salt of: 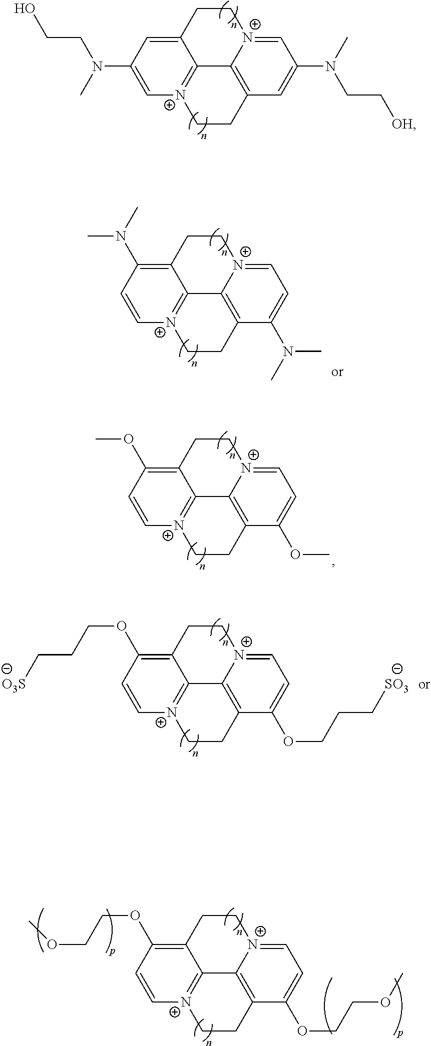
wherein each n is independently 1 or 2, and wherein 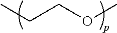
represents an ethylene glycol moiety when p is 1 or a poly(ethylene glycol) moiety when p is greater than 1.

18. The redox flow battery according to claim 13, wherein the one or more compound is a salt of:

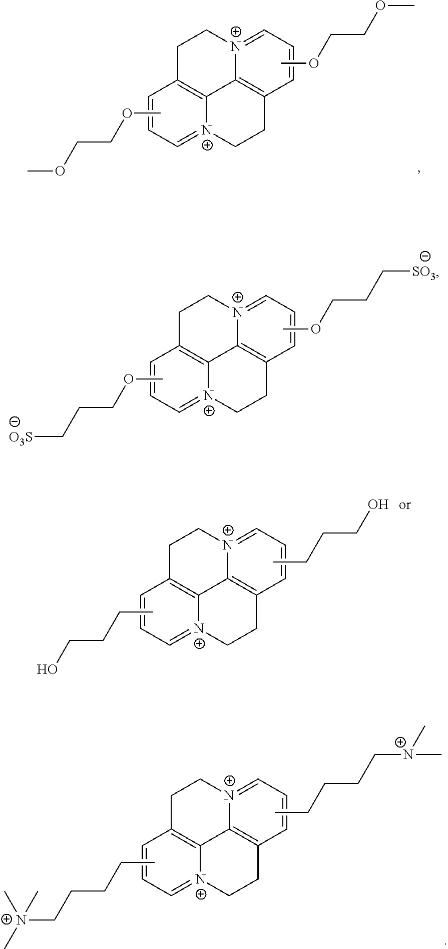

19. An electrochromic material comprising the compound according to claim 1, wherein the compound provides a reversible colour change in all or a portion of the electrochromic material.

20. The electrochromic material of claim 19, which is a window glass (e.g., a smart window), a device display, a reflective blind, a sensor, a mirror, an eyeglass lens, or the like.
